(19) United States
(12) Patent Application Publication
(10) Pub. No.: US 2017/0281175 A1
(43) Pub. Date: Oct. 5, 2017

(54) TISSUE STAPLERS, STAPLER HEADS, AND METHODS OF SURGICALLY FASTENING AND CUTTING A BODILY TISSUE

(71) Applicant: STEPWISE LTD., Tel-Aviv (IL)

(72) Inventors: Simon ROBINSON, Ein Sarid (IL); Yehuda BACHAR, Givaat Shmuel (IL); Ronny WINSHTEIN, Ramat-Hasharon (IL)

(21) Appl. No.: 15/511,467

(22) PCT Filed: Sep. 15, 2015
(86) PCT No.: PCT/IB2015/057083

§ 371 Date: Mar. 15, 2017

Related U.S. Application Data
(60) Provisional application No. 62/050,476, filed on Sep. 15, 2014.

(51) Int. Cl. A61B 17/072; A61B 17/04

(52) U.S. Cl. CPC A61B 17/07207; A61B 17/0469; A61B 2017/07285; A61B 2017/07271; A61B 2017/07257; A61B 2017/07235

(57) ABSTRACT

Staplers and stapler heads for surgically fastening and cutting a bodily tissue or/and organ, including: a stapling jaw configured to include a first stapling jaw portion and a second stapling jaw portion separated from each other by a longitudinal channel. A blade having a sharp edge may be shiftable from a horizontal position to a vertical position so as to allow traveling thereof along the longitudinal channel. First stapling jaw portion may include a first row of tissue fasteners of a first staple size or/and type, and second stapling jaw portion may include a second row of tissue fasteners of a second staple size or/and type. Also described are methods for surgically fastening and cutting a bodily tissue or/and organ.

### RELATED APPLICATION

This application claims the benefit of priority under 35 USC 119(e) of U.S. Provisional Patent Application No. 62/050,476, filed Sep. 15, 2014, entitled “ENDOSCOPIC CUTTER STAPLERS AND METHODS”, the content of which is incorporated herein by reference in its entirety.

### FIELD OF THE INVENTION

The present invention, in some embodiments thereof, relates to surgical tissue staplers and in particular to surgical staplers and endo cutter staplers and to methods of using same during surgeries for sealing and/or cutting a tissue or a bodily organ. Some surgical staplers of the invention are dynamic, being convertible from a non-operational delivery configuration mode, which is applicable for delivery into the body via small-sized passages, to a deployment configuration in which the stapler head is in an operational mode and affords stapling and/or cutting a bodily organ or tissue.

### BACKGROUND OF THE INVENTION

There is a growing trend towards designing surgical tools with smallest possible diameters while keeping, if not also improving, functionality for the surgical practice. In case of minimally invasively introduced laparoscopic tools, smaller diameter may contribute to smaller incisions or cuts made to the skin and soft tissues underneath. In case of natural orifice (e.g., orally) introduction of endoscopic tools, smaller diameter may contribute to the possibility and/or ease to add more tools or other, larger, tools in parallel, via the natural orifice.

In past years this trend also encompasses design efforts of surgical staplers, including disposable or reusable linear staplers and endo cutter staplers. Endoscopic surgical staplers are commonly introduced through laparoscopic ports being 12 mm or more in diameter, so there is a need to provide smaller designs introducible through natural orifices or through laparoscopic ports being 8 mm or less, or even about 6 mm or less in diameter. Some known prior publications focus on new configurations of staples and ways of applying staples (e.g., attaching the staples to and through tissue walls) which allow smaller overall size of the stapler. Such prior publications include: U.S. Pat. Nos. 8,556,935, 8,403,956, 8,365,973, the disclosures of which are fully incorporated herein by reference. However, introducing new staples and methods of anchoring may create unnecessary burden to the surgeons' society and may also increase costs to hospitals and patients.

Therefore, there is a need for surgical staplers and endo cutters having small size during introduction into a patient's body with minimal to no change in current staples and methods of use.

### SUMMARY OF THE INVENTION

The present invention, in some embodiments thereof, relates to surgical tissue stapler heads, and surgical tissue staplers and to methods of use thereof for fastening, sealing or/and cutting bodily tissues or organs.

The surgical tissue staplers and stapler heads of the invention include a stapling jaw and an anvil jaw positionable to oppose the stapling jaw, the stapling jaw configured to include a first stapling jaw portion and a second stapling jaw portion being relatively positioned so as to be oppositely separated from each other by a longitudinal channel extending therebetween. The first stapling jaw portion includes a first row of tissue fasteners of a first staple size or/and type, and the second stapling jaw portion includes a second row of tissue fasteners of a second staple size or/and type. The surgical stapler device of the invention is preferably convertible during surgery from a delivery configuration in which the surgical stapler's head is non-operable to a deployment operative configuration.

Having asymmetric structure with respect to the first and second jaw portions being different in the number of rows loaded with fasteners and/or the size and/or type of fasteners, affords a particular minimal cross section dimension(s) of the surgical tissue heads and staplers. Consequently, the surgical tissue heads and staplers of the invention provide particular minimal invasive surgical means.

In an aspect of some embodiments, there is provided a (e.g., linear) tissue stapler head, which includes a stapling jaw configured to include a first stapling jaw portion and a second stapling jaw portion being relatively positioned so as to be oppositely separated from each other by a longitudinal channel extending therebetween, the longitudinal channel is sized and configured for allowing a blade having a sharp edge to travel therealong; and an anvil jaw positionable to oppose the stapling jaw.

In some embodiments, the first stapling jaw portion includes a first row of tissue fasteners of a first staple size or/and type adjacent to a first side of the longitudinal channel, and the second stapling jaw portion includes a second row of tissue fasteners of a second staple size or/and type adjacent to a second side of the longitudinal channel.

In some embodiments, the tissue fasteners of the first size or/and type or/and of the second size or/and type have staple legs that are plastically deformable to an inwardly bent form.

In some embodiments, the tissue fasteners of the second size or/and type have a minimal leg length being greater than about 3 mm.

In some embodiments, the tissue fasteners of the first size or/and type have a maximal leg length being less than about 3 mm.

In some embodiments, at least one of the tissue fasteners of the first and second size or/and type includes at least one rigid or elastic prong.

In some embodiments, the second stapling jaw portion has a plurality of rows of tissue fasteners including the second row of tissue fasteners of the second staple size or/and type. In some embodiments, the second stapling jaw portion has a number of rows of tissue fasteners which is greater by at least one than the number of rows of the first stapling jaw portion. In some embodiments, the second stapling jaw portion comprises two rows of tissue fasteners and the first stapling jaw portion comprises one row of tissue fasteners. In some embodiments, the second stapling jaw portion comprises three rows of tissue fasteners and the first stapling jaw portion comprises one row of tissue fasteners. In some embodiments, the second stapling jaw portion comprises three rows of tissue fasteners and the first stapling jaw portion comprises two rows of tissue fasteners.

In some embodiments, the first stapling jaw portion includes suturing means.

In some embodiments, the first stapling jaw portion includes tissue bonding means, tissue coagulating means, or tissue adhering means.

In some embodiments, in close proximity between the stapling jaw and the anvil jaw, the first stapling jaw portion faces a first contact surface of the anvil jaw and the second stapling jaw portion faces a second contact surface of the anvil jaw. Optionally, a first distance extending between the first stapling jaw portion and the first contact surface is less than a second distance extending between the second stapling jaw portion and the second contact surface. Optionally, the first contact surface and the second contact surface are distanced apart from each other by a contact surface transition portion located therebetween, the contact surface transition portion includes a rise such that the first contact surface is elevated relative to the second contact surface.

In some embodiments, the tissue stapler head is configured and operative so as to be convertible from a delivery configuration including the stapler head in a non-operational mode and in which the stapler head is passable through a passage enclosing a minimal inner diameter, to a deployment configuration including the stapler head being in an operational mode. Optionally, in the delivery configuration, the stapling jaw and the anvil jaw are consecutively arranged lengthwise relative to each other. Optionally, in the delivery configuration, the stapler head has maximal cross-sectional dimensions including a maximal height, a maximal width, or/and a maximal diameter, each of the dimensions being smaller than the minimal inner diameter of the passage. Optionally, in the delivery configuration, each of the maximal stapler head cross-sectional dimensions is equal to or less than about 8 mm.

In an aspect of some embodiments there is provided a (e.g., linear) tissue stapler, which includes an elongated body having a longitudinal axis, a stapling jaw configured for including a plurality of parallel rows of tissue fasteners, an anvil jaw; and a drive member including a blade having a sharp edge extending between the stapling jaw and the anvil jaw. The blade is optionally shiftable from a horizontal position to a vertical position relative to the longitudinal axis. Optionally, the blade, in the vertical position, is configured to travel along a longitudinal channel extending between oppositely separated portions of the stapling jaw.

In some embodiments, the tissue stapler is configured so as to be selectively convertible from a delivery configuration including a stapler head in a non-operational mode and in which the tissue stapler is passable through a passage enclosing a minimal inner diameter, to a deployment configuration including the stapler head in an operational mode.

In some embodiments, the tissue stapler is configured such that when in the delivery configuration, the blade is in the horizontal position, and when in the deployment configuration, the blade is in the vertical position.

In some embodiments, the tissue stapler is configured such that when in the delivery configuration, the anvil jaw and the stapling jaw are consecutively arranged lengthwise whereby a maximal stapler cross-sectional dimension is less than the minimal inner diameter. In some such embodiments, when in the deployment configuration, the anvil jaw and the stapling jaw are juxtapositionally arranged and form the operational mode stapler head whereby the maximal stapler cross-sectional dimension is equal to or greater than the minimal inner diameter of the passage.

In some embodiments, when converting between the delivery configuration and the deployment configuration, the anvil jaw is actuated for shifting towards the stapling jaw. Optionally, the tissue stapler further includes a pivot around which the anvil jaw swivels during the shifting. Optionally, the pivot is part of the drive member.

In some embodiments, during the shifting, the anvil jaw undergoes a planar displacement being a combination of a planar rotation and a planar translation. Optionally, the planar displacement is effected and maintained via a planar linkage arrangement. Optionally, the planar linkage arrangement includes at least one linkage that includes the blade being rotatable between the horizontal position and the vertical position about a driver-blade joint connecting between the drive member and the blade. Relative rotation between the anvil jaw and the blade may be facilitated about a blade-anvil junction formed between the blade and the anvil jaw.

In some embodiments, the anvil jaw includes a grooved track extending between a proximal side and a distal side thereof below the channel, so as to allow a flange to pass along the grooved track, the flange connects the blade to the anvil jaw to form the blade-anvil junction. Optionally, the blade-anvil junction is positioned proximally to the distal side of the grooved track during the relative rotation between the anvil jaw and the blade when the blade rotates.

In some embodiments, during the conversion of the tissue stapler from the delivery configuration to the deployment configuration, upon the shifting of the blade from the horizontal position to the vertical position and the anvil jaw planar displacement, the stapler is configured such that the anvil jaw shifts distally relative to the blade and thereby opposes the stapling jaw.

In some embodiments, the tissue stapler is configured such that the anvil jaw shifting distally relative to the blade is restricted to motion of the grooved track relative to the flange, whereby the flange is allowed to travel along the grooved track from the blade-anvil junction to a retracted flange location at, or adjacent to, the proximal side of the grooved track. Optionally, the stapler head in the operational mode is configured such that the drive member and the blade are sequentially drivable in a distal direction with the flange along the grooved track from the retracted flange location up to a final flange location located at, or adjacent to, the distal end of the grooved track.

In some embodiments, the tissue stapler is configured to have in the delivery configuration a maximal cross-sectional dimensions including a maximal height, a maximal width and/or a maximal diameter equal to or less than 8 mm.

In an aspect of some embodiments, there is provided a method of surgically fastening or/and cutting a bodily tissue or organ inside a body of a subject. In some embodiments, the method includes at least one of the following steps (not necessarily in same order):
providing a (e.g., linear) tissue stapler head inside the body of the subject, the tissue stapler head includes a stapling jaw configured to include a first stapling jaw portion and a second stapling jaw portion being relatively positioned so as to be oppositely separated from each other by a longitudinal channel extending therebetween;
clasping the bodily tissue or organ between the stapling jaw and an anvil jaw;
passing a blade having a sharp edge along the longitudinal channel to form a cutting line across the bodily tissue or organ;
actuating the stapler head such that the first stapling jaw portion releases a first row of tissue fasteners of a first staple size or/and type along and adjacent a first side of the cutting line, and the second stapling jaw portion releases a second row of tissue fasteners of a second staple size or/and type along and adjacent a second side of the cutting line;

compressing the removable body part via the first row of tissue fasteners, and compressing the remaining body part via the second row of tissue fasteners, such that adjacent the cutting line the removable body part has a smaller thickness than the remaining body part.

In some embodiments, the passing the blade and the releasing the first and second rows of tissue fasteners effects dissecting of the bodily tissue or organ along the cutting line into a removable body part sealed with the first row of tissue fasteners, and a remaining body part sealed with the second row of tissue fasteners.

In some embodiments, the actuating of the stapler head further includes compressing the stapling jaw towards the anvil jaw to thereby inwardly bend staple legs of the tissue fasteners over the bodily tissue or organ.

The tissue stapler head may further include a drive member including the blade, and is configured for driving the blade to travel along the longitudinal channel.

In an aspect of some embodiments there is provided a method of surgically fastening and cutting a bodily tissue or organ inside a body, the method includes at least one of the following steps (not necessarily in same order):
providing a (e.g., linear) tissue stapler head inside the body of the subject, the tissue stapler head includes a stapling jaw configured to include a first stapling jaw portion and a second stapling jaw portion being relatively positioned so as to be oppositely separated from each other by a longitudinal channel extending therebetween;
clasping the bodily tissue or organ between the stapling jaw and an anvil jaw;
actuating the stapler head thereby dividing the bodily tissue or organ into a first body part having a first cut and sealed end and a second body part having a second cut and sealed end, the first cut and sealed end is held by tissue fasteners of a first size or/and type and the second cut and sealed end is held by tissue fasteners of a second size or/and type.

In some embodiments, the dividing the bodily tissue or organ includes stapling the first body part with at least one row of the tissue fasteners of the first size or/and type, and stapling the second body part with at least one row of the tissue fasteners of the second size or/and type.

In an aspect of some embodiments there is provided a method of surgically fastening and cutting a bodily tissue or organ inside a body, the method includes at least one of the following steps (not necessarily in same order):
providing a passage connecting between an ex-vivo environment and an in-vivo location in vicinity of the bodily tissue or organ;
passing a (e.g., linear) tissue stapler through the passage towards the in-vivo location, the tissue stapler includes an elongated body having a longitudinal axis and a stapler head, wherein the stapler head includes a first member configured for including a plurality of parallel rows holding tissue fasteners, a second member, and a drive member including a blade having a sharp edge extending between the first and second members;
effecting emergence, via the passage, of the stapler head out of the body;
operating the tissue stapler so as to effect shifting of the blade from a horizontal position to a vertical position, relative to the longitudinal axis, and to effect releasing of the tissue fasteners from the first member, thereby fastening and cutting the bodily organ or tissue;
providing the tissue stapler configured with the first member includes a first member portion and a second member portion, the first and second member portions being oppositely separated from each other by a longitudinal channel extending therebetween, and wherein during the operating, in the vertical position, the blade travels along the longitudinal channel, or/and providing the tissue stapler configured with the first member portion includes a first row of tissue fasteners, and the second member portion includes a second row of tissue fasteners, and wherein the operating the stapler includes the first member portion releasing a first group of tissue fasteners characterized by a first staple size or/and type, and the second member portion releasing a second group of tissue fasteners characterized by a second staple size or/and type.

In some embodiments, the operating the tissue stapler is followed by converting the tissue stapler from a delivery configuration to a deployment configuration having the stapler head in an operational mode.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1B schematically illustrate full and enlarged partial isometric views of a prior art concept for endo cutter staplers;

FIGS. 1C-1D schematically illustrate cross sectional front views of a ‘3+3’ stapling head and a ‘2+2’ stapling head of prior art concepts for endo cutter staplers;

FIGS. 1E-1F schematically illustrate cross sectional front views of an exemplary ‘3+1’ staple head of a convertible endo cutter stapler, in accordance with some embodiments of the present invention;

FIGS. 2A-2B schematically illustrate a delivery configuration and a deployment configuration, respectively, in side views of an exemplary linear tissue stapler, in accordance with some embodiments of the present invention;

FIGS. 3A-3C schematically illustrate side views of different shifting mechanisms between a delivery configuration and a deployment configuration of exemplary linear tissue staplers, in accordance with some embodiments of the present invention;

FIGS. 4A-4E schematically illustrate side views of different positions in deploying an exemplary linear tissue stapler configured to have its anvil jaw positioned distally to the stapling jaw when in the delivery configuration, in accordance with some embodiments of the present invention;

FIGS. 5A-5C illustrate isometric views of different positions in deploying an exemplary linear tissue stapler configured to have its anvil jaw positioned proximally to the stapling jaw when in the delivery configuration, in accordance with some embodiments of the present invention;

FIGS. 6A-6D illustrate side views of different positions of an anvil jaw of the linear tissue stapler of FIGS. 5A-5C during shifting between a delivery configuration and a deployment configuration, in accordance with some embodiments of the present invention;

FIGS. 7A-7D illustrate side, full or partial, views of the anvil jaw of FIGS. 6A-6D in different positions during movement from a closed position to an open position, in accordance with some embodiments of the present invention;

FIGS. 8A-8E illustrate side, full or partial, views of the stapler head of the linear tissue stapler of FIGS. 5A-5C during cutting and stapling function, in accordance with some embodiments of the present invention;

FIGS. 9A-9D schematically illustrate an exemplary ‘3+1’ stapler head with a single row of tissue fasteners with fasteners of a size and/or type different than fasteners of remaining rows of tissue fasteners, in accordance with some embodiments of the present invention;

FIGS. 10A-10D schematically illustrate another exemplary ‘3+1’ stapler head with a sliding tissue fastening/bonding apparatus, in accordance with some embodiments of the present invention;

FIGS. 11A-11C illustrate the stapler head of FIGS. 8A-8E in isometric-frontal view, front view and cross sectional front view, respectively in accordance with some embodiments of the present invention; and

FIGS. 12A-12G schematically illustrate different scenarios representing possible exemplary steps in a method of deploying a linear tissue stapler and surgically affecting a tissue in a body, in accordance with some embodiments of the present invention.

### DESCRIPTION OF EMBODIMENTS OF THE INVENTION

The present invention, in some embodiments thereof, relates to surgical tissue stapler heads, and surgical tissue staplers and to methods of use thereof for fastening, sealing or/and cutting bodily tissues or organs.

According to some embodiments, the tissue staplers of the invention are configured so as to be selectively convertible from a delivery configuration including a stapler head in a non-operational mode and in which the linear tissue stapler is passable through a passage enclosing a minimal inner diameter, to a deployment configuration including the stapler head in an operational mode.

According to some embodiments, the surgical tissue staplers and stapler heads of the invention are particular minimal in cross sectional dimensions, thus affording introduction into the body in a minimal invasive manner.

As will be explained in more details below, in some embodiments, some particular minimal cross sectional dimensions of the surgical tissue staplers and stapler heads of the invention are afforded by the convertible properties of the surgical tissue heads or/and by the asymmetric structure of the surgical tissue head, being sufficient for allowing delivery thereof via the abovementioned passage.

The following preferred embodiments may be described in the context of exemplary laparoscopic surgical procedures for ease of description and understanding. However, the invention is not limited to the specifically described devices and methods, and may be adapted to various clinical applications without departing from the overall scope of the invention. For example, devices and related methods including concepts described herein may be used for other surgical procedures such as, but not limited to: single-port laparoscopy, endoscopy, and NOTES (“Natural Orifice Translumenal Endoscopic Surgery”) assisted endoscopic or laparoscopic surgeries.

Provided immediately below is a “Definition” section, where certain terms related to the invention are defined specifically.

The term “surgical stapler” as used herein refers to a medical device which is used to place surgical fasteners.

The terms “surgical fastener”, “tissue fastener”, “surgical staples” or “tissue staples”, as used herein are interchangeable and refer to any pronged artifact that can pierce through tissue layers for fixating or bonding them together. A surgical fastener may include one leg or a plurality of legs, possibly linked one with the other via a leg bridge; each leg is pronged at its end and sized to pass through a plurality of soft tissue layers. Surgical fasteners or staples types include, but are not limited to, those that are elastic and capable to bounce from a stressed configuration (e.g., by stretching or compressing its legs open or close) to a nominal less stressed configuration during deployment, those that are rigid enough in order to preclude deformation during deployment, or those that are plastically deformable during deployment, as in the case of surgical tissue staples, where the legs are forced to bend to a fixedly deformed closed configuration, and are commonly used to close wounds or openings in place of sutures.

Surgical staplers may be designed for open surgeries or for endoscopic or laparoscopic surgeries, may or may not include tissue cutting means, and may be disposable or reusable. The surgical staplers are optionally loaded with disposable cartridges containing the fasteners (e.g., staples). The stapler line may be straight, curved or circular.

A surgical stapler as other types of staples can include two opposing jaws pivotally movable one with the other: a “stapling jaw” which houses the cartridge/staples and optionally includes means to push staples to protrude towards the opposing jaw, an “anvil jaw”, having a number of recesses or grooves corresponding in number and position to the staples in “stapling jaw”. Upon grasping and compressing a tissue with the jaws, each staple favorably penetrates through the tissue with each of its two pronged legs which are then inwardly bent as they press against the corresponding anvil recesses.

The terms “linear tissue stapler”, “linear stapler” and “surgical linear stapler” as used herein refer to a surgical stapler loaded with two or more staggered straight rows of staples, commonly used in abdominal surgery, thoracic surgery, gynecology, and pediatric surgery. Linear tissue staplers may be reusable or disposable, with distinctive designs for open surgeries and for endoscopic surgeries.

The terms “endocutter”, “endo-cutter”, “endo cutter stapler” and “cutting stapler” as used herein refer to a stapler with a blade provided between staples rows used for resection and transection of organs or tissues. Common cutting staplers are loaded with two groups of staples rows (each with a single row, or double or triple staggered rows, or more, or any combination thereof) and simultaneously cuts and divides tissue between the two groups of rows in parallel to closing cut tissue ends with staples.

The present invention, in some embodiments thereof, relates to surgical fasteners appliers and in particular to surgical linear staplers and endo cutter staplers.

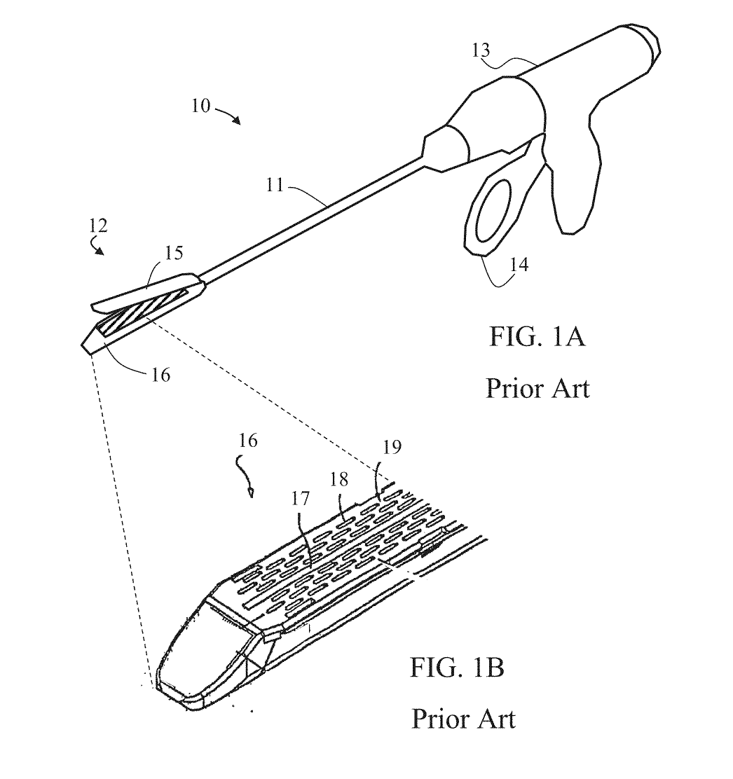

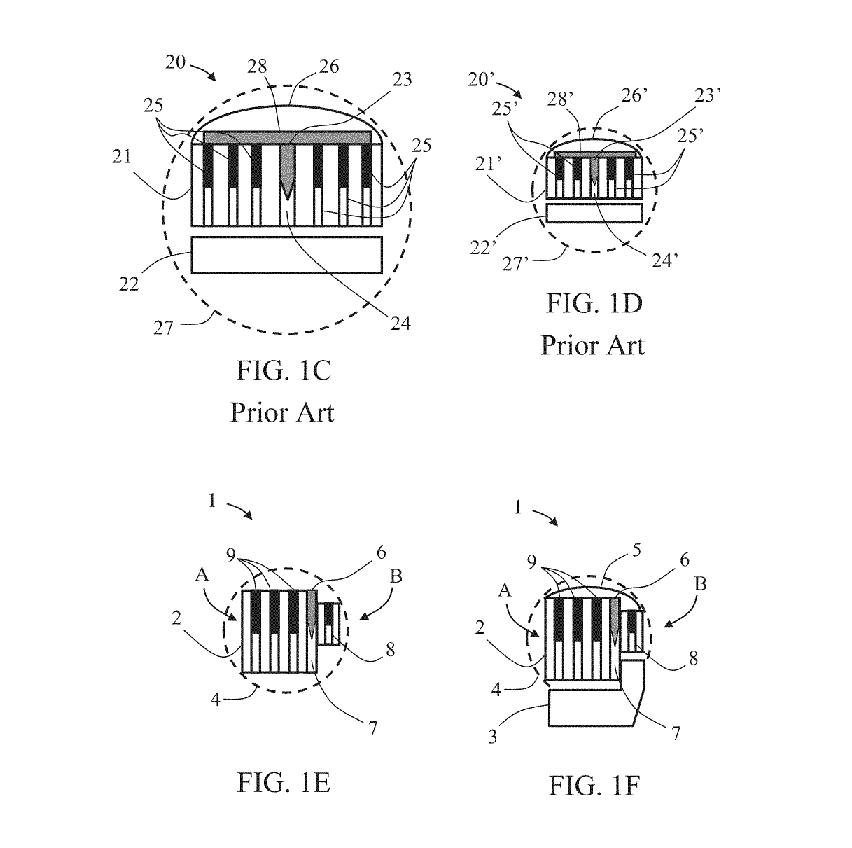

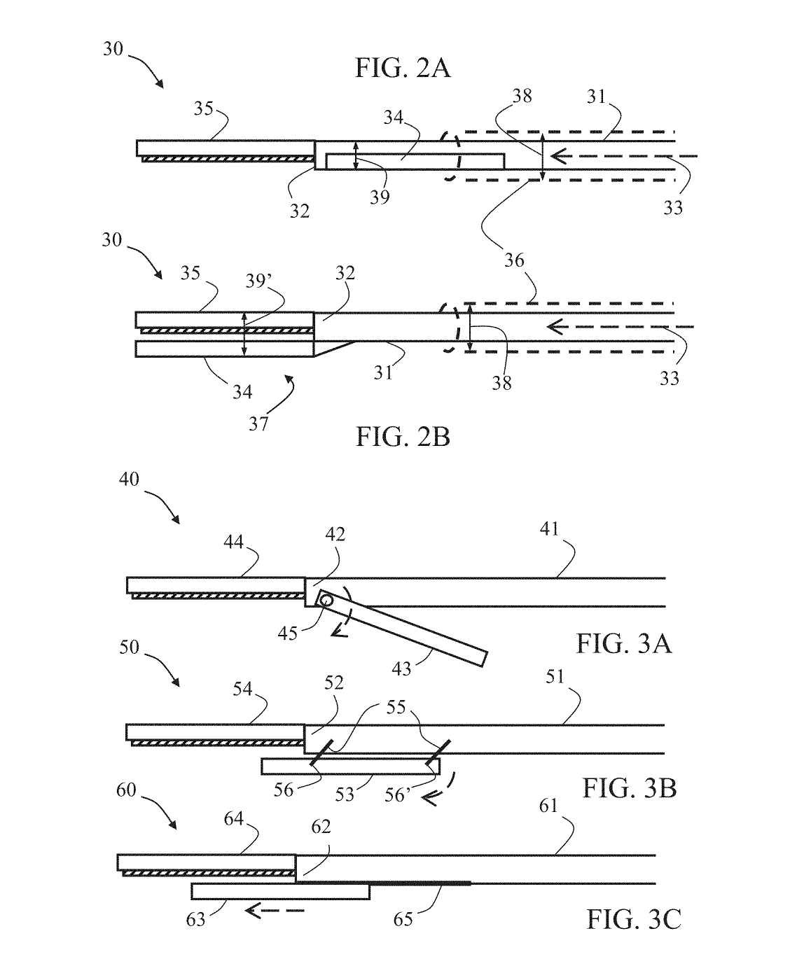

Reference is made to FIG. 1A, which schematically illustrates an isometric view of a prior art linear (cutting) stapler 10, and to FIG. 1B, which schematically illustrates an enlarged partial view of a stapling jaw 16 of cutting stapler 10. Cutting stapler 10 includes an elongated shaft 11 connected at its distal end to a stapler head 12 and at its proximal end to a handle assembly 13 that comprises an arm actuator 14. Head 12 includes a pair of opposing jaws, namely stapling jaw 16 and an anvil jaw 15, which are pivotally movable one with respect to the other and configured to grasp a tissue of allowed thickness therebetween. Stapling jaw 16 includes three rows 19 of staggered grooved slots 18 in each side, separated with an elongated space 17. Each grooved slot 18 houses a tissue staple and a staple pusher, the staple pusher capable of moving vertically in the corresponding grooved slot to press the corresponding tissue staple towards a corresponding depression in anvil jaw 15. A drive member comprising a blade/knife can be forced to pass lengthwise along elongated space 17 and by doing so generates at least one of the following end results: locally clasp adjacent portions of anvil jaw 15 and stapling jaw 16 about a tissue optionally provided therebetween, pushing staples pushers to eject staples toward anvil jaw 15 through the clasped tissue portion, and cut the tissue along elongated space 17 between the jaws, with the blade. Upon actuation of arm actuator 14, the jaws approximate each other and the drive member is pushed distally.

FIG. 1C schematically illustrates cross sectional front view of a ‘3+3’ stapling head 20 of a prior art concept for endo cutter staplers. A ‘3+3’ stapling head design includes a total of six rows of large tissue staples (optionally legs size of 3.5 mm or more) and is commonly used today for securing and removing tissues from sub-abdominal locations. The large staples size is required for penetrating through and bending over relatively thick muscular tissues with avoiding local tissue over-compression, trauma and/or ischemia, while the number of rows in each side of the cutting line and the staggered positioning of the staples between adjacent rows is meant for effective sealing of both ends of the cut tissue along the cutting line. Stapling head 20 includes a stapling jaw 21 and an anvil jaw 22, shown in close approximation, movable one with the other up to maximal opening. Stapling jaw 21 contains a tissue cutting blade 23 provided in a longitudinal channel 24 extending along its length, dividing between two portions each housing three rows of tissue staples 25, staggered one to the other. A fixed cover 26 is provided above stapling jaw, housing the stapling-cutting mechanism that commonly includes a selectively actuated drive member 28 fixedly connected to blade 23 and includes a total of six runners for pushing down the staples 25 sequentially during its travel from a proximal position to a distal position along stapling jaw 21. When jaws are in a closed configuration, stapling head 20 (usually being equal or greater in cross sectional dimension than other parts of the stapler) is sized to be delivered only through large sized endoscopic/laparoscopic channels, such as passage 27, having minimal inner diameter of about 12 mm, or optionally within the range of 10 mm to 14 mm.

FIG. 1D schematically illustrates cross sectional front view of a ‘2+2’ stapling head 20′ of a prior art concept for endo cutter staplers (an exemplary commercially available stapler is the JustRight™ 5 mm Stapler by JustRight Surgical, LLC, from Boulder, Colo., USA). The ‘2+2’ stapling head design includes a total of four rows of small tissue staples (optionally legs size of about 2 mm or less). The small staples size is designed for penetrating through and bending over relatively thin and delicate tissues while avoiding local tissue over-compression, trauma and/or ischemia, while the number of rows in each side of the cutting line and the staggered positioning of the staples between adjacent rows is competent for sealing of both ends of the cut tissue along the cutting line. Stapling head 20′ includes a stapling jaw 21′ and an anvil jaw 22′, shown in close approximation, movable one with the other up to a maximal opening. Stapling jaw 21′ contains a tissue cutting blade 23′ provided in a longitudinal channel 24′ extending along its length, dividing between two portions each housing two rows of tissue staples 25′, staggered one to the other. A fixed cover 26′ is provided above stapling jaw 21′, housing a stapling-cutting mechanism that commonly includes a selectively actuated drive member 28′ fixedly connected to blade 23′ and includes a total of four runners for pushing down the staples 25′ sequentially during its travel from a proximal position to a distal position along stapling jaw 21′. When jaws are in a closed configuration, stapling head 20′ is sized to be delivered through small sized endoscopic/laparoscopic channels, such as passage 27′, having minimal inner diameter of about 6 mm, or optionally within the range of 5 mm to 7 mm.

In an attempt to provide an endo cutter stapler for treating tissues including thick muscular tissues in sub-abdominal locations, yet still allowing passage through small endoscopic channels, the present invention discloses concept of linear (endo cutter) tissue staplers comprising at least one of the following attributes:

1. the stapler is convertible from a delivery configuration, in which the anvil jaw and the stapling jaw are arranged such that the maximal stapler cross-sectional dimension is smaller or less than the minimal inner diameter of a small laparoscopic channel, to a deployment configuration, in which the anvil jaw and the stapling jaw are arranged such that they form an operational stapler head with a maximal cross-sectional dimension equal to or greater than the channel minimal inner diameter.

2. the stapler head is asymmetric in the sense that quantity, type, size or/and distribution of tissue fasteners (e.g., staples) is different from two sides of the blade and elongated channel (e.g., in a form of a ‘2+1’, or ‘3+1’, or ‘3+2’ type). In some such embodiments, stapler head readily incorporates or/and is configured to include a first stapling jaw portion/side and a second stapling jaw portion/side. In some embodiments, the second stapling jaw portion is configured to apply tissue fasteners in similar size and quantity as in large endo cutter staplers, and a first stapling jaw portion/side configured to apply tissue fasteners in much smaller size and/or quantity, thereby decreasing substantially the cross-sectional dimension of the stapler head in corresponding side of the stapler head relative to longitudinal channel. In some embodiments, the stapler head is asymmetric in the sense that at least the rows of tissue fasteners adjacent to each side of the longitudinal channel are different one from the other in the type and/or size of staples. It should be recognized that in surgical procedures involving withdrawal of cut tissues, each of tissue remaining within the body and tissue removed from the body may be treated in a different manner. Specifically, for tissue remaining within the body it is necessary that the cut end would be fastened and sealed in a sufficient manner that eliminate untightened spaces yet loose enough to exclude over-compression and necrosis. For the removed tissue part, the cut end may be fastened in a much less strict manner in terms of untightened spaces, and that would allow further compression of the tissue to a minimal thickness, hence even a single row with smaller staples may be used. The surgical tissue stapler and stapler head of the invention, being of ‘2+1’, or ‘3+1’, or ‘3+2’ type provides a solution to those surgical requirements. It should be further recognized that the particular stapler's head type as disclosed herein affords uniquely minimal invasive delivery into the body.

3. in order to further minimize its size during delivery, the stapling jaw cover can be provided retracted (proximally) and configured to slide distally over the staples rows. The cover can be provided fixated to the blade and runners therefore functioning as a drive member instead of housing a driver mechanism slidable therein.

Thus, according to one aspect of the present invention, there is provided a linear tissue stapler head, comprising: a stapling jaw configured to include a first stapling jaw portion and a second stapling jaw portion being relatively positioned so as to be oppositely separated from each other by a longitudinal channel extending therebetween, the longitudinal channel is sized and configured for allowing a blade having a sharp edge to travel therealong; and an anvil jaw positionable to oppose the stapling jaw; wherein the first stapling jaw portion includes a first row of tissue fasteners of a first staple size or/and type adjacent to a first side of the longitudinal channel, and the second stapling jaw portion includes a second row of tissue fasteners of a second staple size or/and type adjacent to a second side of the longitudinal channel.

According to some embodiments, the second stapling jaw portion has a plurality of rows of tissue fasteners including the second row of tissue fasteners of the second staple size or/and type. According to some embodiments, a plurality of rows of tissue fasteners is two rows or three rows. First and second stapling jaw portions are optionally different one from the other with respect to at least the staples size or/and type of the rows adjacent to each side of the longitudinal channel. Namely, at least the single row immediately adjacent one side of the longitudinal channel is different than the other single row immediately adjacent the other side of the longitudinal channel, such that each of these two rows comprises a different size/type of tissue fasteners. According to some embodiments, first and second stapling jaw portions are different one from the other with respect the entire size or/and type of staples. In some embodiments, the second stapling jaw portion has a number of rows of tissue fasteners which is greater by at least one, than the number of rows of the first stapling jaw portion. In some embodiments, the second stapling jaw portion has a number of rows of tissue fasteners which is greater by one or two than the number of rows of the first stapling jaw portion. In some embodiments, the second stapling jaw portion has two rows of tissue fasteners of the second staple size or/and type and the first stapling jaw portion has one row of tissue fasteners of a first staple size or/and type. In some embodiments, the second stapling jaw portion has three rows of tissue fasteners of the second staple size or/and type and the first stapling jaw portion has one row of tissue fasteners of a first staple size or/and type. In some embodiments, the second stapling jaw portion has three rows of tissue fasteners of the second staple size or/and type and the first stapling jaw portion has two rows of tissue fasteners of a first staple size or/and type.

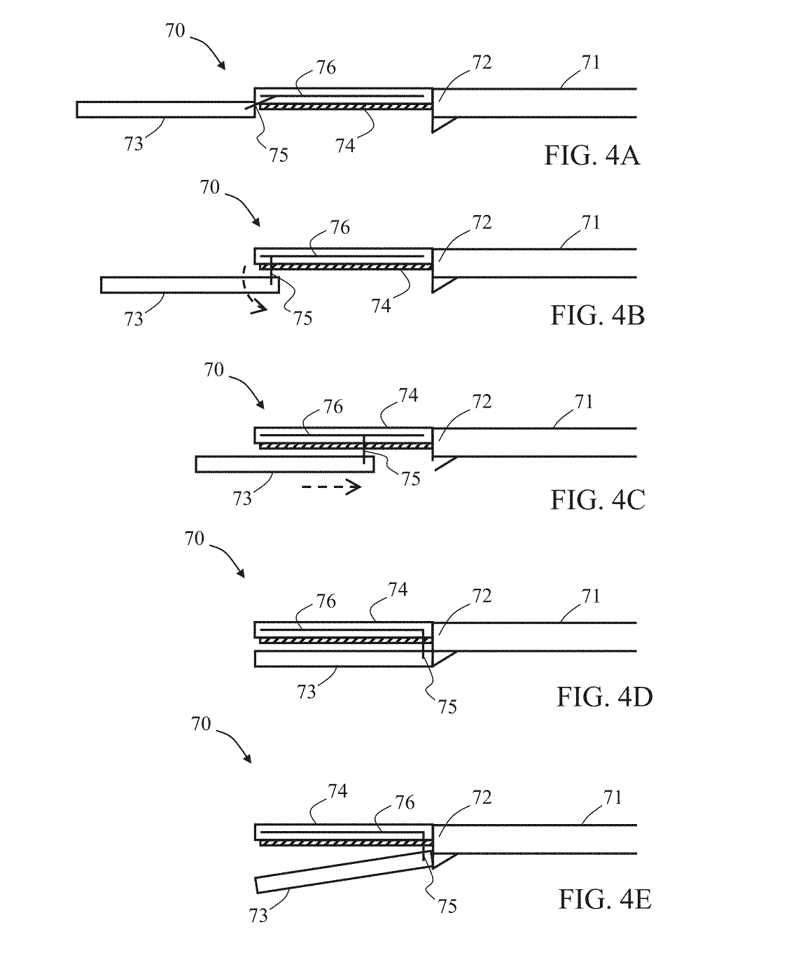

Reference is made to FIGS. 1E-1F which schematically illustrate cross sectional front views of an exemplary ‘3+1’ linear tissue staple head 1 of a convertible endo cutter stapler, in delivery configuration and in deployment configuration, respectively, in accordance with some embodiments of the present invention. Staple head 1 includes a stapling jaw 2 and an anvil jaw 3. The linear tissue stapler is convertible from a delivery configuration (FIG. 1E), in which stapler head 1 is not formed and not operational, to a deployment configuration (FIG. 1F) in which stapler head 1 is formed and operational. When in the delivery configuration, anvil jaw 3 and stapling jaw 2 are consecutively arranged lengthwise (as, for example, shown in FIG. 2A), optionally one is behind the other, such that the maximal stapler cross-sectional dimension is smaller than a minimal inner diameter 4 enclosed by a passage such as that formed by a laparoscopic channel.

When in the deployment configuration, anvil jaw 3 and stapling jaw 2 are juxtapositionally arranged and oppose each other, one above the other, forming the operational stapler head such that the maximal stapler cross-sectional dimension is greater than or equal to minimal inner diameter 4. Minimal inner diameter 4 may be about 8 mm or less, optionally about 6 mm or less, optionally about 4 mm or less, or higher, or lower, or an intermediate size. Each possibility represents a separate embodiment of the invention. Stapling head 1 maximal cross-sectional dimension in the deployment configuration may be a maximal height, a maximal width and/or a maximal diameter, and is optionally equal to or greater than about 5 mm, optionally equal to or greater than about 7 mm, optionally equal to or greater than about 8 mm, optionally equal to or greater than about 12 mm, or higher, or lower, or an intermediate value. Each possibility represents a separate embodiment of the invention.

Stapling jaw 2 is configured to include a first stapling jaw portion B and a second stapling jaw portion A being relatively positioned so as to be oppositely separated from each other by a longitudinal channel 7 extending therebetween. First stapling jaw portion B includes a first row of tissue fasteners and second stapling jaw portion A includes a second row of tissue fasteners, optionally adjacent/closest to longitudinal channel 7, out of a number of rows of tissue fasteners (not necessarily of same size or/and type). First stapling jaw portion B includes a first row of tissue fasteners of a first staple size or/and type and is positioned adjacent to a first side of longitudinal channel 7. Second stapling jaw portion A includes a second row of tissue fasteners of a second staple size or/and type and is positioned adjacent to a second side of the longitudinal channel 7. In the figures, stapling jaw 2 includes a total of four rows of tissue staples, and a drive member 5 in the form of a slidable cover provided with a blade 6 having a sharp edge. Drive member 5 is configured to travel along longitudinal channel 7 extending therebetween the separated opposing portions of stapling jaw 2. In some embodiments and as shown, first stapling jaw portion B contains a single row of small tissue fasteners 8 and second stapling jaw portion A contains three staggered rows of large tissue staples 9. First stapling jaw portion B may contain a single row of small tissue fasteners 8 and second stapling jaw portion A may contain two staggered rows of large tissue staples 9. Optionally, the number of small tissue staples 8 is greater than the number of large tissue staples 9 per each row, optionally being further compacted together along the row. In some embodiments, the tissue fasteners of the second row of tissue fasteners have a second size or/and type of tissue fasteners. In some embodiments, the second size or/and type of tissue fasteners have a minimal leg length being greater than about 3 mm. In some embodiments, the tissue fasteners of the first row of tissue fasteners have a first size or/and type of tissue fasteners. In some embodiments, the first size or/and type of tissue fasteners have a maximal leg length being less than about 3 mm.

A minimal leg size of the large tissue staples 9 legs is optionally greater than 3 mm in length, optionally about 3.5 mm, or optionally about 3.8 mm, or optionally about 4.1 mm. A maximal leg size of the small tissue staples 8 legs is optionally smaller than 3 mm in length, optionally about 2.5 mm or optionally about 2 mm. Optionally, additionally or alternatively, a minimal leg size of large tissue staples 9 legs is optionally equal to or greater than 1.5 mm, optionally equal to or greater than 2 mm, in length, whereas a maximal leg size of small tissue staples 8 legs is optionally smaller by at least 0.2 mm, optionally by at least 0.5 mm, optionally by at least 1 mm, optionally by at least 1.5 mm, than the minimal leg size of large tissue staples 9.

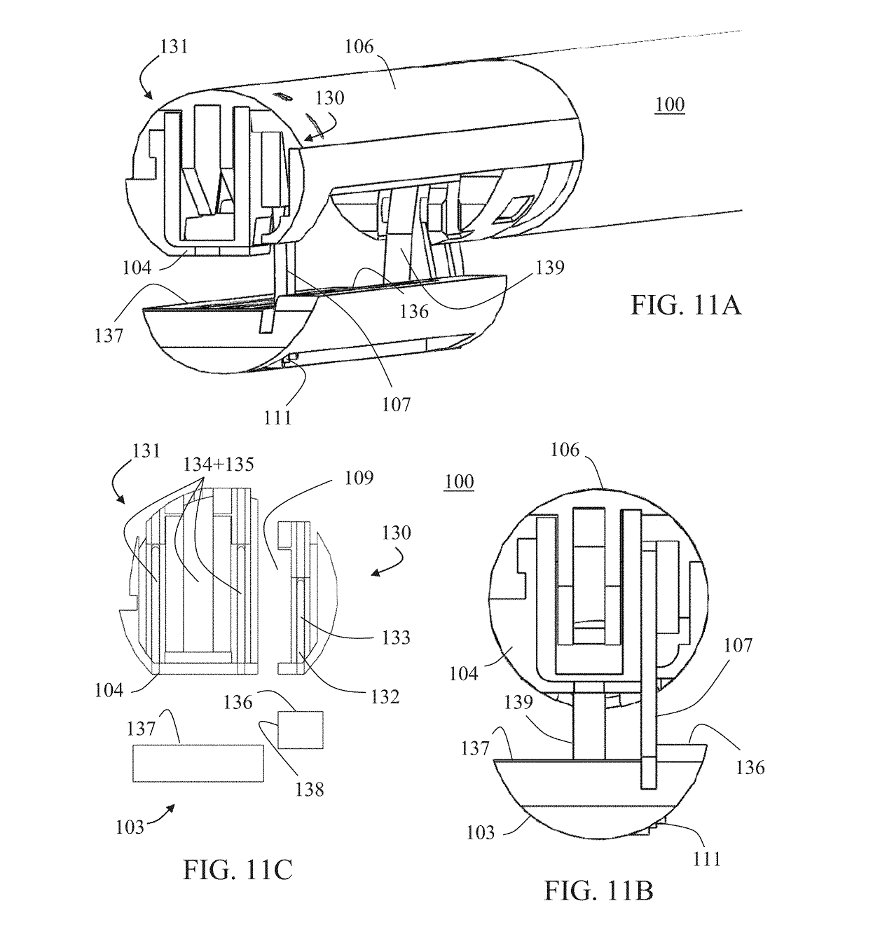

Drive member 5 comprises a number of runners (as, for example, runners 121 shown in FIG. 8C), each runner is slidable along a corresponding grooved route provided along a length of the corresponding stapling jaw portions, A or B. The runner of stapling jaw portion B is optionally shorter and/or held lower than runners of stapling jaw portion A, corresponding to size and level of the packed small tissue staples 8 with respect to those of packed large tissue staples 9. Each row of tissue fasteners includes a number of grooved slots, each grooved slot houses a staple pusher such that upon sliding of the sliding runners distally through the grooved routes, each of the staple pushers moves vertically in the corresponding grooved slot to press the corresponding tissue staple towards a corresponding depression in anvil jaw 3. Anvil Jaw 3 is also shaped accordingly, asymmetrically, such that the contact surface in front of stapling jaw portion A is leveled lower than the contact surface in front of stapling jaw portion B.

FIGS. 2A-B schematically illustrate an exemplary delivery configuration and a deployment configuration, respectively, in side views of an exemplary linear tissue stapler 30, in accordance with some embodiments of the present invention. Linear tissue stapler 30 includes an elongated shaft 31 extending along a longitudinal axis 33 and having a distal end 32, an anvil jaw 34, and a stapling jaw 35 configured for including a plurality of parallel rows of tissue fasteners (e.g., rows 19 of staples shown in FIG. 1B). When in a fully operational mode, the anvil jaw 34 is pivotally connected to elongated body 31 or to stapling jaw 35, and is operable with jaws actuating means, optionally provided proximally to elongated body 31.

Linear tissue stapler 30 is convertible from a delivery configuration (shown in FIG. 2A) to a deployment configuration (shown in FIG. 2B). Optionally, when converting between the delivery configuration and the deployment configuration, anvil jaw 34 is actuated for shifting towards (positionable to oppose) stapling jaw 35. When in the delivery configuration, linear tissue stapler 30 is sized to pass through a passage 36 enclosing a minimal inner diameter 38. Passage 36 may be, for example, a lumen enclosed by a laparoscopic sheath, port or trocar, or, optionally and alternatively, a lumen enclosed by an endoscopic channel. The minimal inner diameter may be equal to or less than about 20 mm, optionally, equal to or less than about 12 mm, optionally, equal to or less than about 8 mm, optionally, equal to or less than about 6 mm, optionally, equal to or less than about 4 mm, or higher, or lower, or an intermediate size. Each possibility represents a separate embodiment of the invention. As shown, anvil jaw 34 and stapling jaw 35 are consecutively arranged lengthwise whereby a maximal stapler cross-sectional dimension 39 is smaller than minimal inner diameter 38. When in the delivery configuration, anvil jaw is positioned entirely proximally to shaft distal end 32. Consequently, stapler 30 is absent of an operational stapler head (i.e., it does not have a functional head capable of performing at least one of grasping, cutting and stapling to a body tissue).

When in the deployment configuration (FIG. 2B), anvil jaw 34 is positioned mostly or entirely distally to shaft distal end 32 and is juxtapositionally arranged with stapling jaw 35 to form an operational stapler head 37. In the deployment configuration the anvil jaw 34 overlaps (i.e., coincides partially or wholly) with stapling jaw 35. Stapling jaw 35 is optionally coupled to shaft distal end 32 and lying distally thereto in both delivery configuration and deployment configuration. Anvil jaw optionally interlocks with shaft distal end 32 and/or with stapling jaw 35 in the deployment configuration to form operational stapler head 37 which is distally adjacent shaft distal end 32. When in the form of operational stapler head 37, the stapling jaw 35 and anvil jaw 34 are movable with respect to each other from an open position, wherein the jaws are spaced apart, to a closed position (shown in FIG. 2B), wherein the jaws are in close proximity to one another.

The maximal stapler cross-sectional dimension 39′ in the deployment configuration is equal to or greater than minimal inner diameter 38. Cross-sectional dimensions 39 and 39′ may be a maximal height, a maximal width and/or a maximal diameter. The maximal stapler cross-sectional dimension in the deployment configuration is equal to or greater than about 3 mm, optionally equal to or greater than about 5 mm, optionally equal to or greater than about 8 mm, optionally equal to or greater than about 12 mm, optionally equal to or greater than about 20 mm, optionally equal to or greater than about 30 mm, or higher, or lower, or an intermediate value. Each possibility represents a separate embodiment of the invention.

The maximal stapler or stapling head cross-sectional dimensions in the delivery configuration may be a maximal height, a maximal width and/or a maximal diameter. The maximal stapler cross-sectional dimension in the delivery configuration is equal to or less than about 20 mm, optionally equal to or less than about 15 mm, optionally equal to or less than about 10 mm, optionally equal to or less than about 8 mm, optionally equal to or less than about 6 mm, optionally equal to or less than about 4 mm, or lower, or an intermediate value.

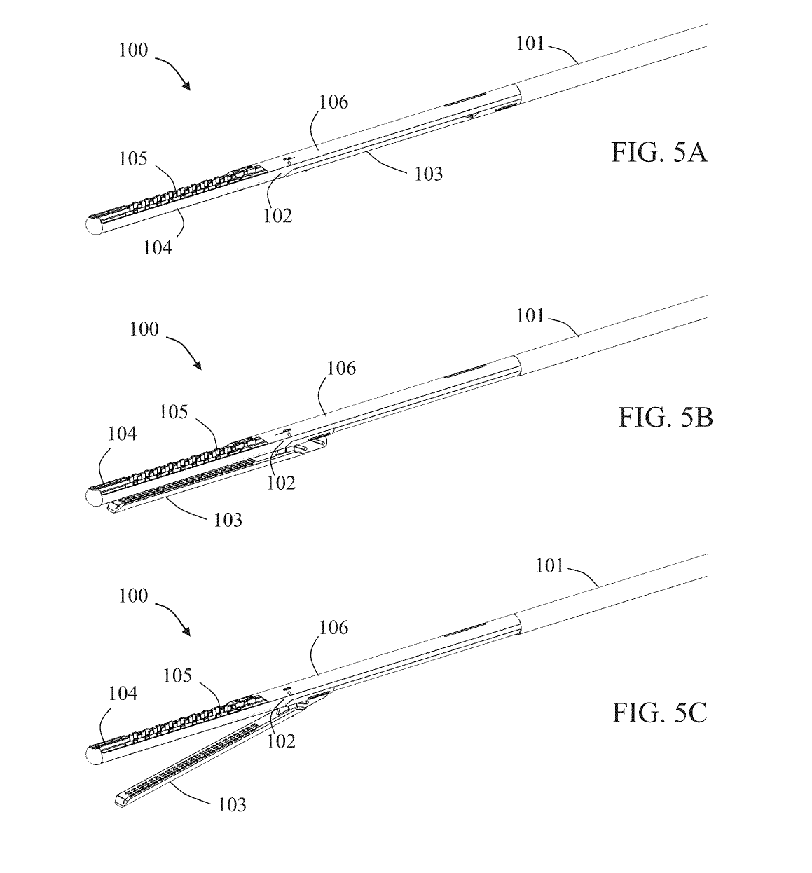

Reference is made to FIGS. 3A-3C which schematically illustrate side views of exemplary different shifting mechanisms between a delivery configuration and a deployment configuration of exemplary linear tissue staplers 40, 50 and 60, in accordance with some embodiments of the present invention. Linear tissue stapler 40 (FIG. 3A) includes an elongated shaft 41 with a shaft distal end 42, an anvil jaw 43, and a stapling jaw 44 containing parallel rows of tissue fasteners (e.g., similar to rows 19 as shown in FIG. 1B). Linear tissue stapler 40 is convertible from a delivery configuration (similar to that shown in FIG. 2A) to a deployment configuration (similar to that shown in FIG. 2B). When in the delivery configuration, linear tissue stapler 40 is sized to pass through a passage enclosing a minimal inner diameter (e.g., similar to passage 36 as shown in FIG. 2A), such as a lumen enclosed by a laparoscopic sheath, port or trocar, or an endoscopic channel. The minimal inner diameter may equal to or less than about 20 mm, optionally equal to or less than about 12 mm, optionally equal to or less than about 8 mm, optionally equal to or less than about 6 mm, optionally equal to or less than about 4 mm or less, or higher, or lower, or an intermediate size. Each possibility represents a separate embodiment of the present invention. Anvil jaw 43 and stapling jaw 44 may be consecutively arranged lengthwise whereby their maximal cross-sectional dimension is smaller than minimal inner diameter. When in the delivery configuration, anvil jaw 43 may be positioned entirely proximally to shaft distal end 42 making stapler 40 absent of an operational stapler head. When in the deployment configuration, anvil jaw 43 is positioned mostly or entirely distally to shaft distal end 42 and is juxtapositionally arranged with stapling jaw 44 to form an operational stapler head. In the deployment configuration the anvil jaw 43 may oppose and/or overlap with stapling jaw 44.

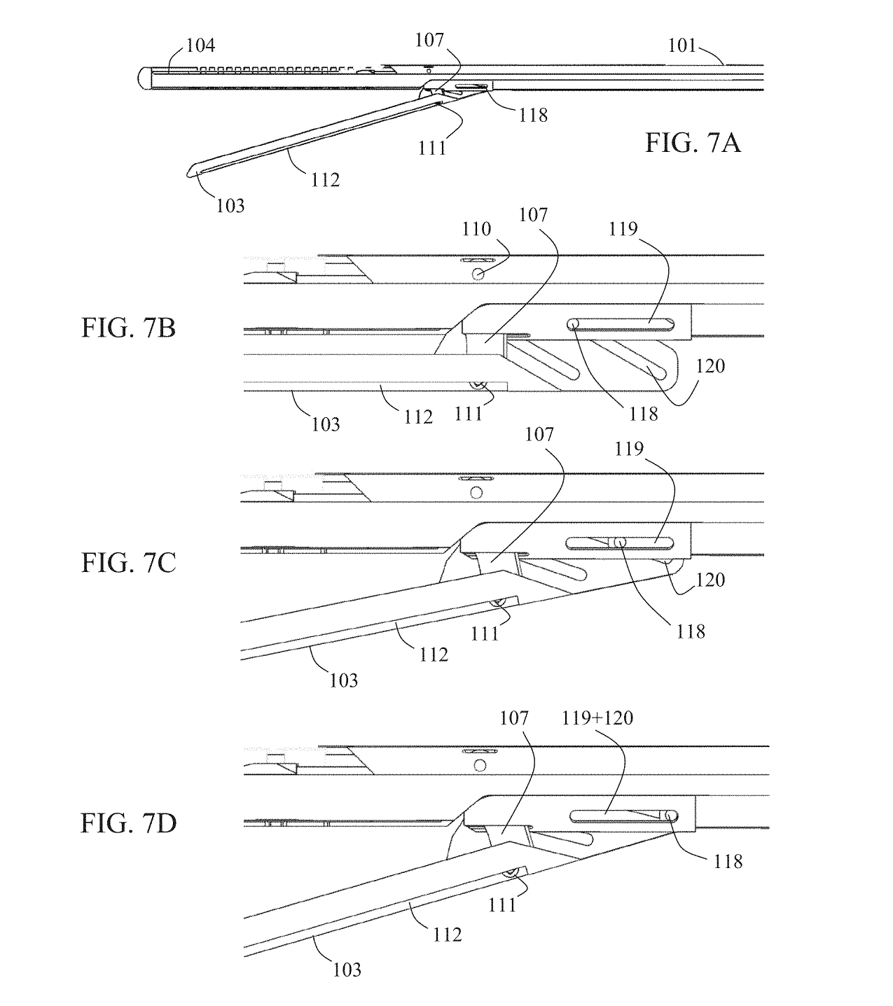

Stapling jaw 44 is optionally coupled to shaft distal end 42 and lying distally thereto in both delivery configuration and deployment configuration. Anvil jaw 43 is configured to swivel towards stapling jaw 44 when shifting between the delivery configuration and the deployment configuration as shown in FIG. 3A. Swiveling may be actuated about a pivot point provided at or adjacent shaft distal end 42, optionally facilitated by a hinge 45, as shown. Optionally, the pivot is part of a drive member (as, for example, drive member 106 shown in FIG. 5A-5C). Anvil jaw 43 may then interlock with shaft distal end 42 and/or with stapling jaw 44 in the deployment configuration to form the operational stapler head distally adjacent shaft distal end 42. Stapling jaw 44 and anvil jaw 43 are then movable with respect to each other from an open position, wherein the jaws are spaced apart, to a closed position, wherein the jaws are in close approximation to one another. The maximal stapler cross-sectional dimension in the deployment configuration is equal to or greater than minimal inner diameter, and may be a maximal height, a maximal width and/or a maximal diameter. The maximal stapler cross-sectional dimension in the deployment configuration is equal to or greater than about 3 mm, optionally equal to or greater than about 5 mm, optionally equal to or greater than about 8 mm, optionally equal to or greater than about 12 mm, optionally equal to or greater than about 20 mm, optionally equal to or greater than about 30 mm, or higher, or lower, or an intermediate value. Each possibility represents a separate embodiment of the invention.

Linear tissue stapler 50 (FIG. 3B) comprises an elongated shaft 51 with a shaft distal end 52, an anvil jaw 53, and a stapling jaw 54 containing parallel rows of tissue fasteners (e.g., similarly to rows 19 of staples shown in FIG. 1B). Linear tissue stapler 50 is convertible from a delivery configuration (similar to that shown in FIG. 2A) to a deployment configuration (similar to that shown in FIG. 2B). When in the delivery configuration, linear tissue stapler 50 is sized to pass through a passage enclosing a minimal inner diameter (as, for example, minimal inner diameter 38 shown in FIG. 2A), such a lumen enclosed by a laparoscopic sheath, port or trocar, or an endoscopic channel. The minimal inner diameter may be equal to or less than about 20 mm optionally equal to or less than about 12 mm, optionally equal to or less than about 8 mm, optionally equal to or less than about 6 mm, optionally equal to or less than about 4 mm, or higher, or lower, or an intermediate size. Each possibility represents a separate embodiment of the invention. Anvil jaw 53 and stapling jaw 54 may be consecutively arranged lengthwise whereby their maximal cross-sectional dimension is smaller than minimal inner diameter. When in the delivery configuration, anvil jaw 53 may be positioned entirely proximally to shaft distal end 52 making stapler 50 absent of an operational stapler head. When in the deployment configuration, anvil jaw 53 is positioned mostly or entirely distally to shaft distal end 52 and is juxtapositionally arranged with stapling jaw 54 to form an operational stapler head. In the deployment configuration the anvil jaw 53 may oppose and/or overlap with stapling jaw 54.

Stapling jaw 54 is optionally coupled to shaft distal end 52 and lying distally thereto in both delivery configuration and deployment configuration. Anvil jaw 53 is configured to revolve around a point 56 located thereon on the shifting as shown in FIG. 3B. Optionally, anvil jaw 53 is further configured to translate relative to stapling jaw 54 on the shifting, optionally during or after revolving. Revolving may be actuated about a plurality of pivot points, such as points 56 and 56′, provided along anvil jaw 53 and/or along shaft 51. Optionally, revolving is part of a planar translation motion based on a closed chain linkage motion, optionally “four-bar-linkage” being its simplest form structure (i.e., constructed from four links connected in a loop by four one degree of freedom joints), comprising the followings as four linkages: anvil jaw 53, part of shaft 51 and two opposing revolving bars 55. Optionally, the four-bar linkage is configured and dimensioned to act as a parallelogram linkage having two opposing linkages identical in size. Anvil jaw 53 may then interlock with shaft distal end 52 and/or with stapling jaw 54 in the deployment configuration to form the operational stapler head distally adjacent shaft distal end 52. Stapling jaw 54 and anvil jaw 53 are then movable with respect to each other from an open position, wherein the jaws are spaced apart, to a closed position, wherein the jaws are in close approximation to one another. The maximal stapler cross-sectional dimension in the deployment configuration is equal to or greater than minimal inner diameter, and may be a maximal height, a maximal width and/or a maximal diameter. The maximal stapler cross-sectional dimension in the deployment configuration is equal to or greater than about 3 mm, optionally equal to or greater than about 5 mm, optionally equal to or greater than about 8 mm, optionally equal to or greater than about 12 mm, optionally equal to or greater than about 20 mm, optionally equal to or greater than about 30 mm, or higher, or lower, or an intermediate value. Each possibility represents a separate embodiment of the invention.

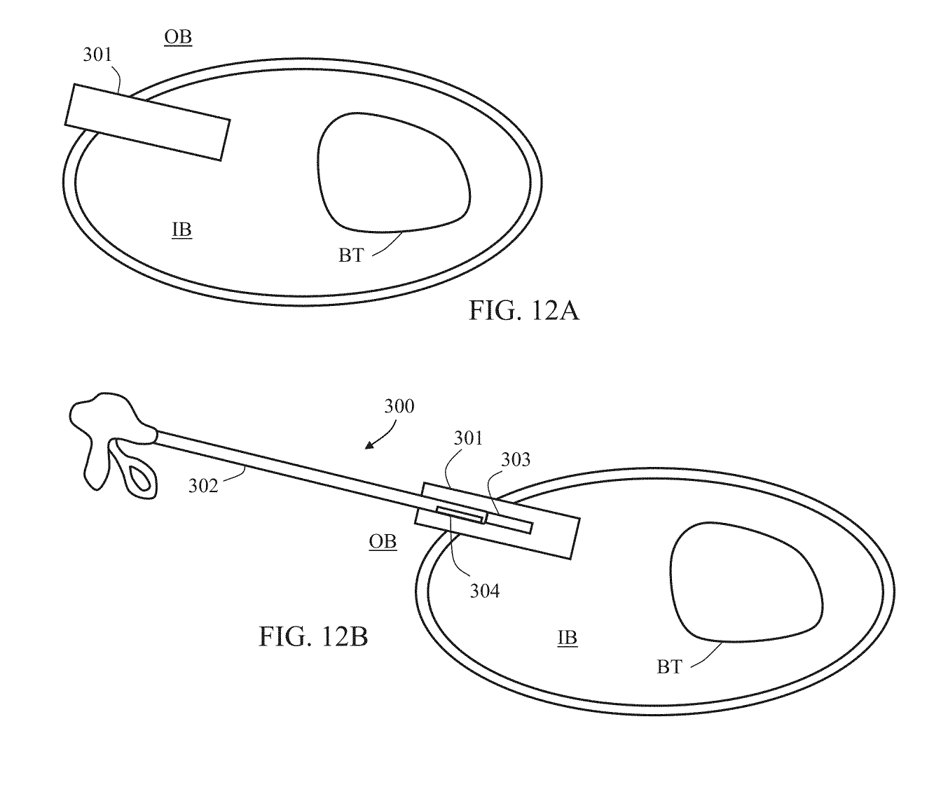

Linear tissue stapler 60 (FIG. 3C) comprises an elongated shaft 61 with a shaft distal end 62, an anvil jaw 63, and a stapling jaw 64 containing parallel rows of tissue fasteners (as, for example, rows 208 and 206 shown in FIG. 9A). Linear tissue stapler 60 is convertible from a delivery configuration (similarly to as shown in FIG. 2A) to a deployment configuration (similarly to as shown in FIG. 2B). When in the delivery configuration, linear tissue stapler 60 is sized to pass through a passage enclosing a minimal inner diameter (as, for example, minimal inner diameter 38 shown in FIG. 2A), such a lumen enclosed by a laparoscopic sheath, port or trocar, or an endoscopic channel. The minimal inner diameter may be equal to or less than about 20 mm, optionally equal to or less than about 12 mm, optionally equal to or less than about 8 mm, optionally equal to or less than about 6 mm, optionally equal to or less than about 4 mm, or higher, or lower, or an intermediate size. Each possibility represents a separate embodiment of the invention. Anvil jaw 63 and stapling jaw 64 may be consecutively arranged lengthwise whereby their maximal cross-sectional dimension is smaller than minimal inner diameter. When in the delivery configuration, anvil jaw 63 may be positioned entirely proximally to shaft distal end 62 making stapler 60 absent of an operational stapler head. When in the deployment configuration, anvil jaw 63 is positioned mostly or entirely distally to shaft distal end 62 and is juxtapositionally arranged with stapling jaw 64 to form an operational stapler head. In the deployment configuration the anvil jaw 63 may oppose and/or overlap with stapling jaw 64.

Stapling jaw 64 is optionally coupled to shaft distal end 62 and lying distally thereto in both delivery configuration and deployment configuration. Anvil jaw 63 is configured to slide proximally and/or distally when shifting between the delivery configuration and the deployment configuration, for example along a track 65 provided thereon or along part of shaft 61. Anvil jaw 63 may then interlock with shaft distal end 62 and/or with stapling jaw 64 in the deployment configuration to form the operational stapler head distally adjacent shaft distal end 62. Stapling jaw 64 and anvil jaw 63 are then movable with respect to each other from an open position, wherein the jaws are spaced apart, to a closed position, wherein the jaws are in close approximation to one another. The maximal stapler cross-sectional dimension in the deployment configuration is equal to or greater than minimal inner diameter, and may be a maximal height, a maximal width and/or a maximal diameter. The maximal stapler cross-sectional dimension in the deployment configuration is equal to or greater than about 3 mm, optionally equal to or greater than about 5 mm, optionally equal to or greater than about 8 mm, optionally equal to or greater than about 12 mm, optionally equal to or greater than about 20 mm, optionally equal to or greater than about 30 mm, or higher, or lower, or an intermediate value. Each possibility represents a separate embodiment of the invention.

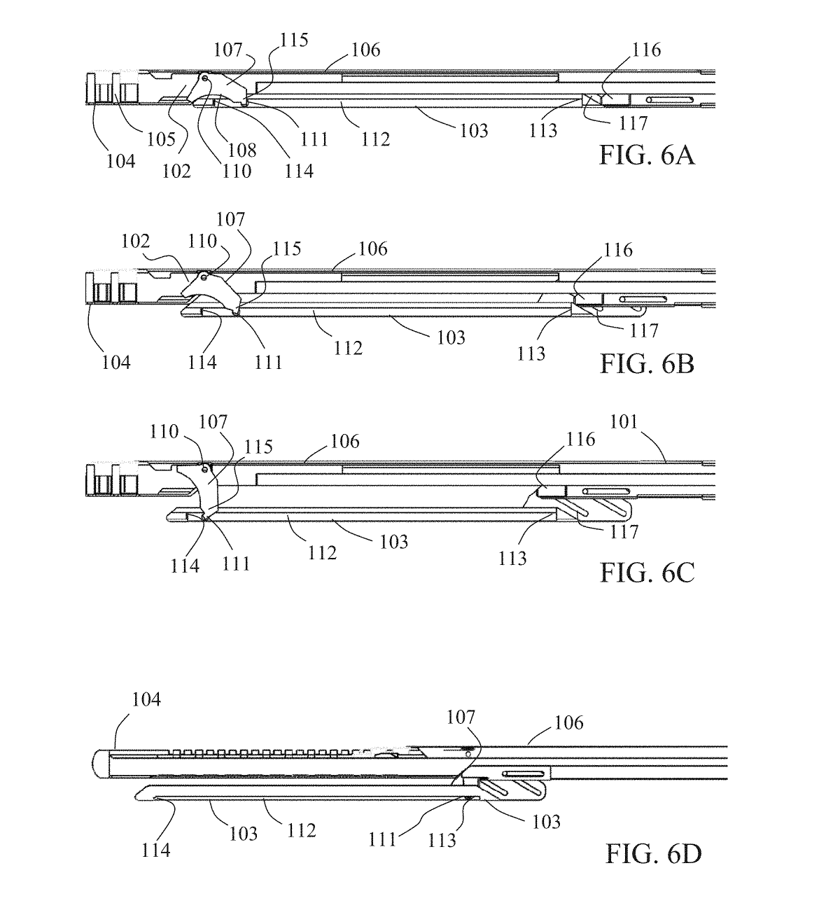

FIGS. 4A-4E schematically illustrate side views of different positions in deploying an exemplary linear tissue stapler 70 configured to have its anvil jaw positioned distally to the stapling jaw when in the delivery configuration, in accordance with some embodiments of the present invention. Linear tissue stapler 70 comprises an elongated shaft 71 with a shaft distal end 72, an anvil jaw 73, and a stapling jaw 74 containing parallel rows of tissue fasteners (e.g., rows 206 and 208 of shown in FIG. 9A). Linear tissue stapler 70 is convertible from a delivery configuration (shown in FIG. 4A) to a deployment configuration (shown in FIG. 4D). When in the delivery configuration, linear tissue stapler 70 is sized to pass through a passage enclosing a minimal inner diameter (not shown), such a lumen enclosed by a laparoscopic sheath, port or trocar, or an endoscopic channel. The minimal inner diameter may be 20 mm or less, optionally about 12 mm or less, optionally about 8 mm or less, optionally about 6 mm or less, optionally about 4 mm or less, or higher, or lower, or an intermediate size. Anvil jaw 73 and stapling jaw 74 may be consecutively arranged lengthwise whereby maximal cross-sectional dimension of stapler 70 is smaller than minimal inner diameter. When in the delivery configuration, anvil jaw 73 is positioned mostly or entirely distally to stapling jaw 74. When in the deployment configuration, anvil jaw 73 is juxtapositionally arranged with stapling jaw 74 to form an operational stapler head, optionally opposing and/or overlapping with stapling jaw 74.

Stapling jaw 74 is optionally coupled to shaft distal end 72 and lying distally thereto in both delivery configuration and deployment configuration. Anvil jaw 73 is configured to swivel towards stapling jaw 74 (as shown in FIGS. 4A-4B) when shifting between the delivery configuration and the deployment configuration. Swiveling may be actuated about a pivot point provided at or adjacent distal end of stapling jaw 74, optionally facilitated by a hinge or a rotating arm 75, as shown. The shifting towards deployment configuration is optionally followed by proximal sliding motion of anvil jaw 73, as shown in FIG. 4C, optionally along a slot or track 76, optionally provided along stapling jaw 74, optionally using rotating arm 75 coupled to track 76 with a runner (not shown). Anvil jaw 73 may then interlock with shaft distal end 72 and/or with stapling jaw 74 in the deployment configuration to form the operational stapler head distally adjacent shaft distal end 72 (FIG. 4D). Stapling jaw 74 and anvil jaw 73, once forming an operational stapling head, are movable with respect to each other from an open position (FIG. 4E), wherein the jaws are spaced apart, to a closed position (FIG. 4D), wherein the jaws are in close approximation to one another. The maximal stapler cross-sectional dimension in the deployment configuration is equal to or greater than minimal inner diameter, and may be a maximal height, a maximal width and/or a maximal diameter. The maximal stapler cross-sectional dimension in the deployment configuration is equal to or greater than about 3 mm, optionally equal to or greater than about 5 mm, optionally equal to or greater than about 8 mm, optionally equal to or greater than about 12 mm, optionally equal to or greater than about 20 mm, optionally equal to or greater than about 30 mm, or higher, or lower, or an intermediate value. Each possibility represents a separate embodiment of the invention.

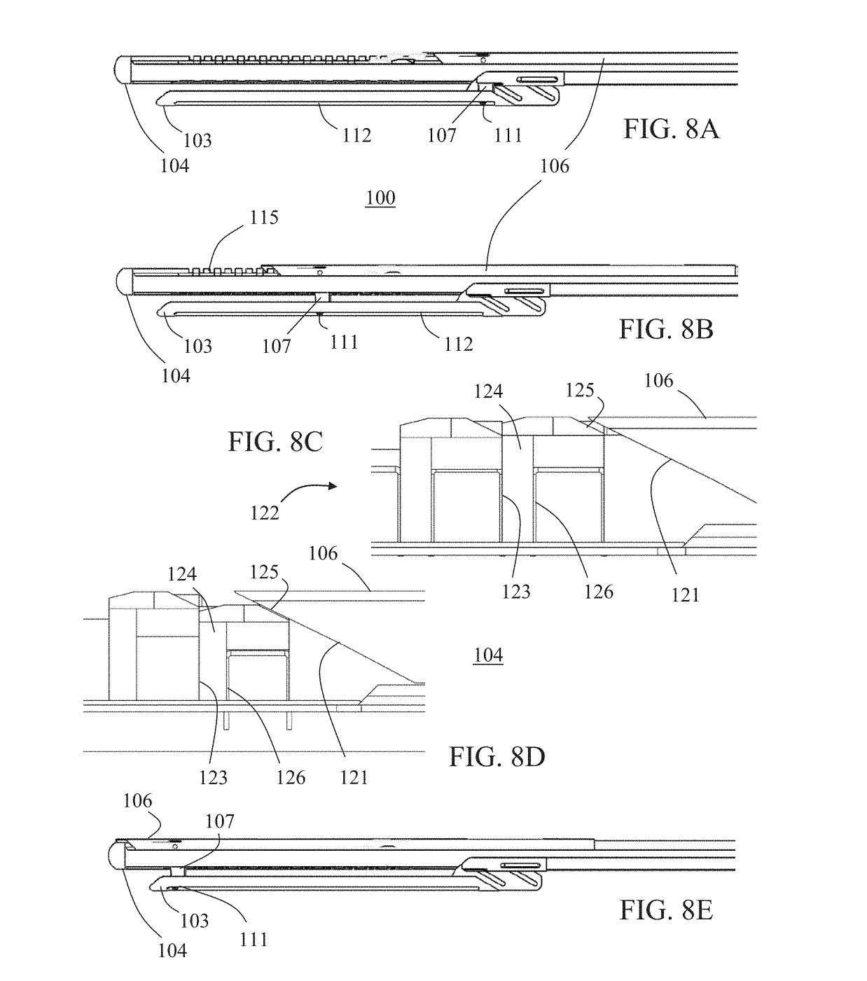

Reference is now made to FIGS. 5A-5C which illustrate isometric views of different positions in deploying an exemplary linear tissue stapler 100 configured to have its anvil jaw positioned proximally to the stapling jaw when in its delivery configuration, in accordance with some embodiments of the present invention. Linear tissue stapler 100 comprises an elongated shaft 101 with a shaft distal end 102, an anvil jaw 103, and a stapling jaw 104 containing parallel rows 105 of tissue fasteners. Linear tissue stapler 100 is convertible from a delivery configuration (as shown in FIG. 5A) to a deployment configuration (as shown in FIG. 5B). When in the delivery configuration, linear tissue stapler 100 is sized to pass through a passage enclosing a minimal inner diameter (not shown), such a lumen enclosed by a laparoscopic sheath, port or trocar, or an endoscopic channel. The minimal inner diameter may be equal to or less than about 20 mm, optionally equal to or less than about 12 mm, optionally equal to or less than about 8 mm, optionally equal to or less than about 6 mm, optionally equal to or less than about 4 mm, or higher, or lower, or an intermediate size. Each possibility represents a separate embodiment of the invention. Anvil jaw 103 and stapling jaw 104 are consecutively arranged lengthwise (anvil jaw 103 is positioned behind or proximal to stapling jaw 104) whereby their maximal cross-sectional dimension is smaller than minimal inner diameter. When in the delivery configuration, anvil jaw 103 is positioned entirely proximally to shaft distal end 102 making stapler 100 absent of an operational stapler head. When in the deployment configuration, anvil jaw 103 is positioned mostly distally to shaft distal end 102 and is juxtapositionally arranged partially overlapping with stapling jaw 104 to form an operational stapler head. A drive member 106 in the form of a slidable cover is shown in a fully retracted position distally to stapling jaw 104. Drive member 106 is provided with a blade 107 having a sharp edge 108 extending between the jaws (shown in FIGS. 6A-6D).

Stapling jaw 104 is an extension of shaft distal end 102, optionally coupled thereto or unitary with it, and lying distally thereto in both delivery configuration and deployment configuration. When shifting between the delivery configuration and the deployment configuration, anvil jaw 103 goes through a planar translation derived from a parallelogram linkage coupling mechanism and then undergoes a sliding motion until docking in a predetermined posture below stapling jaw 104. Anvil jaw 103 may then interlock with shaft distal end 102 and/or with stapling jaw 104 to form an operational stapler head. Stapling jaw 104 and anvil jaw 103 are then movable with respect to each other from an open position (shown in FIG. 5C), wherein the jaws are spaced apart, to a closed position (shown in FIG. 5B), wherein the jaws are in close approximation to one another. The maximal stapler cross-sectional dimension in the deployment configuration may be a maximal height, a maximal width and/or a maximal diameter. The maximal stapler cross-sectional dimension in the deployment configuration is equal to or greater than about 3 mm, optionally equal to or greater than about 5 mm, optionally equal to or greater than about 8 mm, optionally equal to or greater than about 12 mm, optionally equal to or greater than about 20 mm, optionally equal to or greater than about 30 mm, or higher, or lower, or an intermediate value. Each possibility represents a separate embodiment of the invention.

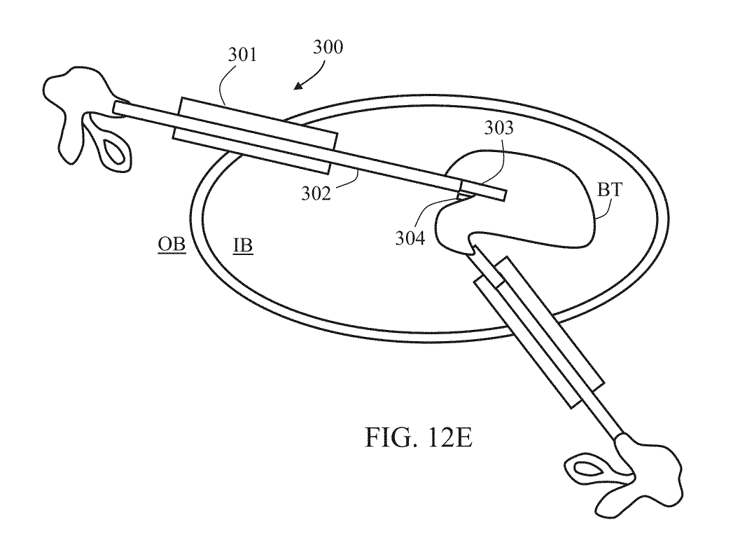

FIGS. 6A-6D illustrate side views of different positions of anvil jaw 103 during its shifting from the delivery configuration to the deployment configuration. Blade 107 is shiftable from a horizontal position (shown in FIG. 6A) when in the delivery configuration to a vertical position (shown in FIG. 6C) when in the deployment configuration, relative to the longitudinal axis of shaft 101. FIG. 6B shows an intermediate location of anvil jaw 103 during shifting from the delivery configuration to the deployment configuration. Blade 107 when in its vertical position, is configured to travel along a longitudinal channel 109 (shown in FIGS. 11A-C) between separated opposing portions of stapling jaw 104.

Upon shifting from the delivery configuration to the deployment configuration, anvil jaw 103 undergoes a planar displacement, which combines a planar rotation and a planar translation. The planar displacement is effected and maintained by a planar linkage arrangement in which blade 107 performs as one of its linkages, and anvil jaw 103 performs as a second linkage. Blade 107 is rotatable between the horizontal position and the vertical position about a driver-blade joint 110 connecting between drive member 106 and blade 107. A relative rotation between anvil jaw 103 and blade 107 is facilitated about a blade-anvil junction 111 provided therebetween.

Anvil jaw 103 includes a grooved track 112, extending between a proximal side 113 and a distal side 114 thereof. Grooved track 112 allows a blade flange 115 to pass there along. Blade flange 115 connects blade 107 to anvil jaw 103 to form a blade-anvil junction 111, which is stationed proximally to distal side 114 of grooved track 112 and throughout which most or all the relative rotation between anvil jaw 103 and blade 107 during blade rotation occurs. Upon the shifting of blade 107 to the vertical position and the planar displacement of anvil jaw 103, the stapler is configured such that anvil jaw 103 shifts distally relative to blade 107 until complete docking in opposition to the stapling jaw 104, as shown in FIG. 6D. Such shifting distally of anvil jaw 103 relative to blade 107 is restricted to motion of grooved track 112 relative to blade flange 115, whereby blade flange 115 is allowed to travel along grooved track 112 to thereby shift from blade-anvil junction 111 to a retracted flange location at or adjacent to proximal side 113 of grooved track 112.

In some embodiments, stapler 100 is configured such that shifting from delivery configuration to deployment configuration includes anvil jaw 103 shifting distally relative to blade 107, thereby opposing stapling jaw 104. Shifting from delivery configuration to deployment configuration is optionally actuated by pushing an actuator flange 116 to travel distally along a length of shaft 101. Actuator flange 116 is coupled to anvil jaw 103 through a first inclined slot 117. In the delivery configuration, actuator flange 116 is located at the lower end of slot 117. Upon forcing actuator flange 116 to move distally, anvil jaw 103 pushes blade 107 to rotate up to the vertical position which in turn forces the distal portion of anvil jaw 103 to move downwards in parallel to its distal motion. Anvil jaw 103 is kept substantially parallel to shaft 101 during its planar translation due to the orientation and dimension of slot 117 thus facilitating a chosen inclined upward motion of actuator flange 116 therealong.

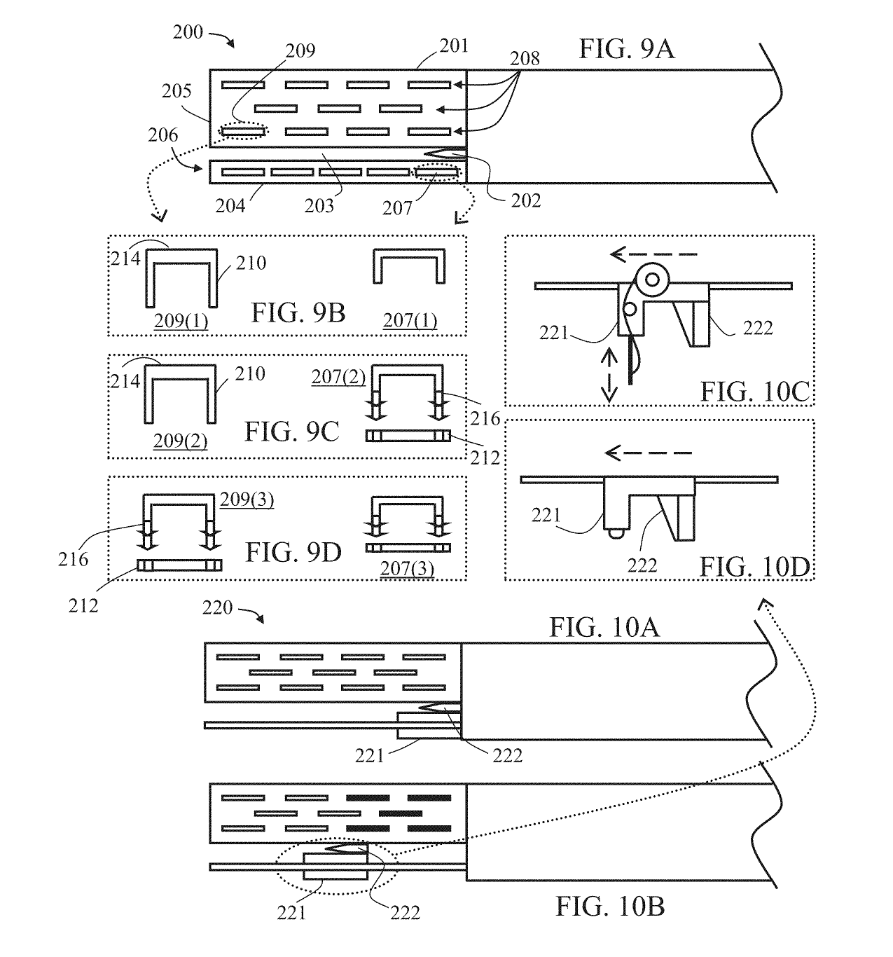

FIGS. 7A-7D illustrate side, full or partial, views of anvil jaw 103 in different positions during movement from a closed position to an open position. Anvil jaw 103 opening is accomplished by pulling a grasping flange 118 to travel proximally along a horizontal passage 119 in shaft 101 from a distal to a proximal position therein (as shown in FIG. 7B). Grasping flange 118 is coupled to anvil jaw 103 through a second inclined slot 120. Upon forcing grasping flange 118 to move proximally through both horizontal passage 119 and inclined slot 120, it also forces them to align with each other, while rotating anvil jaw 103, until inclined slot 120 is horizontal and coincides with horizontal passage 119 (as shown in FIG. 7D). FIG. 7C shows an intermediate position of grasping flange 118 in the middle of horizontal passage 119 and inclined slot 120, whereas anvil jaw 103 is partly opened.

FIGS. 8A-8E illustrate side, full or partial, views of the stapler head of linear tissue stapler 100 during cutting and stapling function (a cut/stapled tissue is absent for simplifying the description). As shown especially in FIGS. 8A and 8B, drive member 106 is actuated to travel distally thereby also forcing blade 107 to travel distally. The functional stapler head is configured such that blade 107 can be driven sequentially in a distal direction with blade flange 115 sliding along grooved track 112 from a retracted flange location up to a final flange location located at or adjacent to distal side 114 of grooved track 112. If a tissue is grasped between the jaws, blade 107 is configured to cut it with sharp edge 108 while moving distally. FIG. 8E shows driving member 106 and blade 107 at their distal-most location.

FIGS. 8C and 8D are side cut views demonstrating in greater details the stapling mechanism upon drive member 106 distal progression. Drive member 106 comprises a number of runners 121, each runner is slidable along a corresponding grooved route 122 provided along a length of stapling jaw 104. Each row of tissue fasteners includes a number of grooved slots 123, each grooved slot houses a staple pusher 124 with a cam head 125. Upon sliding of the sliding runners 121 distally through grooved routes 122, each staple pusher 124 moves vertically in the corresponding grooved slot 123 to press a corresponding tissue staple 126 towards a corresponding depression in anvil jaw 103 (as shown in FIG. 8D).

In some embodiments of the present disclosure, there is provided a linear tissue stapler head comprising a stapling jaw, an anvil jaw, and a drive member provided with a blade having a sharp edge, the blade extending between the jaws. The drive member is configured to travel along a longitudinal channel between separated opposing portions of the stapling jaw. The longitudinal channel is sized and configured for allowing a blade having a sharp edge to travel therealong. The stapling jaw comprises a first stapling jaw portion containing a single row of tissue fasteners (e.g., similar to single row 206 shown in FIG. 9A) and a second stapling jaw portion containing a plurality of rows of tissue fasteners (e.g., similar to plurality of rows 208 shown in FIG. 9A). In some such embodiments, the first stapling jaw portion is configured to apply tissue fasteners of a first size and/or type and the second stapling jaw portion is configured to apply tissue fasteners of a second size and/or type, upon fastening a tissue.

Reference is made to FIGS. 9A-9D which schematically illustrate an exemplary ‘3+1’ stapler head 200 which includes a first stapling jaw portion 204 comprising a single row of first tissue fasteners 207 and a second stapling jaw portion 205 with second tissue fasteners 209, wherein first tissue fasteners 207 are different in size and/or type than second tissue fasteners 209, in accordance with some embodiments of the present invention. FIG. 9A schematically illustrates an upper cross-sectional view of stapling jaw 201, showing a blade 202 slidable in a longitudinal channel 203 extending between first stapling jaw portion 204 and second stapling jaw portion 205 of stapling jaw 201. First stapling jaw portion 204 contains a single row 206 of first tissue fasteners 207, whereas second stapling jaw portion 205 contains remaining three rows 208 of second tissue fasteners 209. Upon distal travel of a driving member (not shown) over both portions 204 and 205, blade 202 fixated thereto is configured to cut through a tissue grasped by the jaws, while runners provided with the driving member actuates stapling mechanism which sequentially presses corresponding staples to penetrate through a grasped tissue and bend back over it. First and second tissue fasteners 207 and 209, respectively include legs 210 being contacted through bridge 214. The first tissue fasteners 207 are in a first size and/or type different than the second tissue fasteners 209 being in a second size and/or type. Tissue fasteners 207 or/and 209 may be of surgical staples type, optionally ‘B-shaped’, optionally made from Titanium or stainless steel or other medical/surgical grade alloy or other material.

FIG. 9B demonstrates a first possible scenario for dissimilar tissue fasteners, showing a first tissue fastener 207(1) configured for stapling via single row 206 of first stapling jaw portion 204, and a second tissue fastener 209(1) configured for stapling via remaining rows 208 of second stapling jaw portion 205. In this example, both fasteners are tissue staples having staples legs and staples bridges 214 that connect the staples legs 210, the staples are plastically deformable to an inwardly bent form following compressing thereof between the jaws. First tissue fastener 207(1) is substantially smaller than second tissue fastener 209(1) having shorter legs (although it can be similar or identical in staple's bridge length). An optional minimal leg size of the second tissue fastener 209(1) may be, for example, greater than about 3 mm in length, optionally about 3.5 mm, or optionally about 3.8 mm, or optionally about 4.1 mm, in length. Each possibility represents a separate embodiment of the invention. In contrast, a leg size of the first tissue fastener 207(1) may be smaller than about 3 mm in length, optionally about 2.5 mm or optionally about 2 mm. Each possibility represents a separate embodiment of the invention. Optionally, additionally or alternatively, a minimal leg size of second tissue fasteners 209(1) legs is optionally equal to or greater than about 1.5 mm, optionally equal to or greater than about 2 mm, in length, whereas a maximal leg size of first tissue fasteners 207(1) legs is optionally smaller by at least 0.2 mm, optionally by at least 0.5 mm, optionally by at least 1 mm, optionally by at least 1.5 mm, than the minimal leg size of second tissue fasteners 209(1). Each possibility represents a separate embodiment of the invention.

FIG. 9C demonstrates a second possible scenario for dissimilar tissue fasteners, showing a first tissue fastener 207(2) configured for stapling via single row 206 of first stapling jaw portion 204, and a second tissue fastener 209(2) configured for stapling via remaining rows 208 of second stapling jaw portion 205. In this example, both fasteners are not necessarily different in size although second tissue fastener 209(2) has plastically deformable legs as described above, while first tissue fastener 207(2) includes at least one rigid or elastic prong 216 which is configured for fixation within corresponding holes in a support member 212, upon stapling. FIG. 9D demonstrates a third possible scenario in which both staples include rigid and/or elastic prongs while being substantially different in leg size, similarly to as described in the example of FIG. 9B.

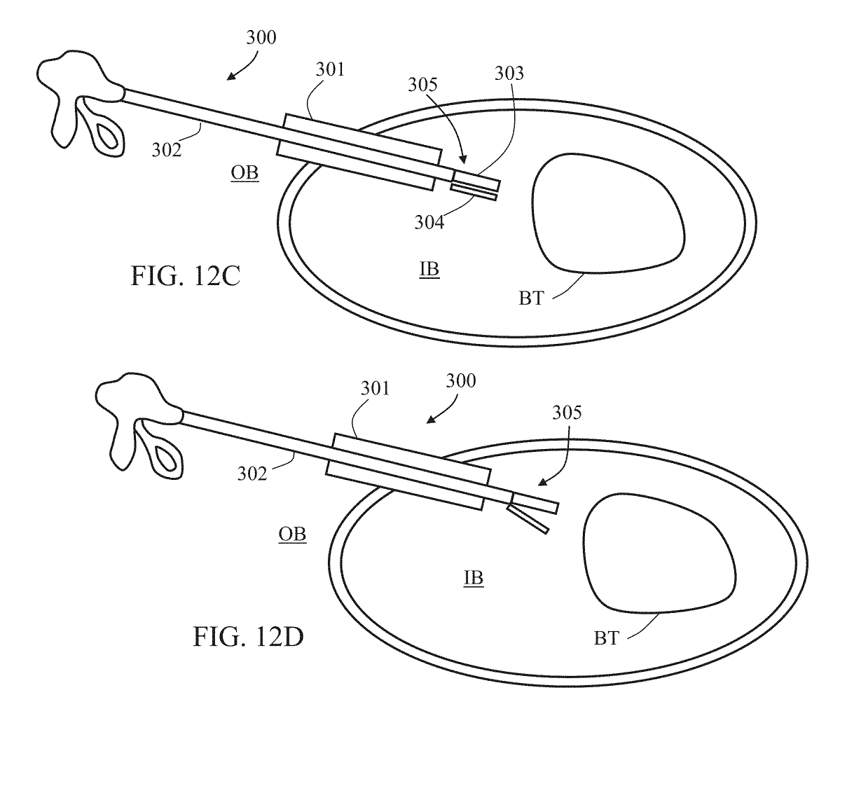

As previously mentioned, in typical instances where a surgical procedure involves withdrawal of cut tissues, it is of primarily importance that the tissue remained within the body would be fastened and sealed at the cut end in an efficient manner, and in a manner that prevents over compression and necrosis, while the removed tissue part can be subjected to less efficient tissue preserving fixating procedures. The present surgical stapler fulfils those requirements which are commonly met in medicinal practice. FIGS. 10A-10D schematically illustrate another exemplary ‘3+1’ stapler head 220 with a sliding tissue fastening/bonding apparatus 221, in accordance with some embodiments of the present invention. In this example, apparatus 221 is fixedly connected to a blade 222 and a driving member (not shown) so that it travels under same progress rate and extent. FIG. 10A shows apparatus 221 and blade 222 at their initial position before stapling and FIG. 10B shows them in midst of stapling cycle. FIG. 10C schematically illustrates a first scenario in which the tissue fasteners of the first size and/or type include suturing means. FIG. 10D schematically illustrates a second scenario in which the tissue fasteners of the first size and/or type include tissue bonding means, such as coagulating or adhering means. Optionally, first stapling jaw portion includes the suturing means.

Optionally, first stapling jaw portion includes the tissue bonding means, tissue coagulating means, or tissue adhering means.

As used herein the terms “tissue bonding means”, “tissue coagulating means”, or “tissue adhering means” are interchangeable and relate to means or procedures used to stop bleeding. Suitable tissue bonding means include, but are not limited to means within which argon gas or high frequency energy may pass or means of hot probes.

FIGS. 11A-11C illustrate the stapler head portion of linear tissue stapler 100 in isometric-frontal view, front view and cross sectional front view, respectively. Drive member 106 is shown in distal-most position together with blade 107, possibly following completion of a tissue stapling and cutting cycle. Blade 107 is shown within longitudinal channel 109 extending between a first stapling jaw portion 130 and a second stapling jaw portion 131 of stapling jaw 104. First stapling jaw portion 130 contains a single row 132 of first tissue fasteners 133, whereas second stapling jaw portion 131 contains remaining three rows 134 of second tissue fasteners 135. Upon distal travel of a driving member 106 over both portions 131 and 132, blade 107 is configured to cut through a tissue grasped by the jaws, while runners 121 actuates stapling mechanism which sequentially presses corresponding staples to penetrate through a grasped tissue and bend back over it. The first tissue fasteners 133 are in a first size which is different than the second tissue fasteners 135 having in a second size. Both fasteners are tissue staples having staples legs plastically deformable to an inwardly bent form following compressing thereof between the jaws. First tissue fasteners 133 are substantially smaller than second tissue fastener 135 having shorter legs (although it can be similar or identical in stapler length). A minimal leg size of the second tissue fastener 135 is greater than 3 mm in length, optionally about 3.5 mm, or optionally about 3.8 mm, or optionally about 4.1 mm, in length. Each possibility represents a separate embodiment of the invention. In contrast, a maximal leg size of the first tissue fastener 133 is smaller than 3 mm in length, optionally about 2.5 mm or optionally about 2 mm. Each possibility represents a separate embodiment of the invention. Optionally, additionally or alternatively, a minimal leg size of second tissue fasteners 135 legs is optionally equal to or greater than 1.5 mm, optionally equal to or greater than 2 mm, in length, whereas a maximal leg size of first tissue fasteners 133 legs is optionally smaller by at least 0.2 mm, optionally by at least 0.5 mm, optionally by at least 1 mm, optionally by at least 1.5 mm, than the minimal leg size of second tissue fasteners 135. Each possibility represents a separate embodiment of the invention.

When stapling jaw and anvil jaw are in the closed position or are in close proximity, as shown, first stapling jaw portion 130 faces a first contact surface 136 of anvil jaw 103 and second stapling jaw portion 131 faces a second contact surface 137 of anvil jaw 103. A first distance, extending between first stapling jaw portion 130 and first contact surface 136 is smaller or less than a second distance, being the distance extending between second stapling jaw portion 131 and second contact surface 137, due to the smaller size of staples 133 of first stapling jaw portion 130 as to remaining staples of second stapling jaw portion 131. According to some embodiments, the first contact surface and the second contact surface are distanced apart from each other by a contact surface transition portion located therebetween. Contact surface transition portion between first contact surface 136 and second contact surface 137 includes a rise 138 such that the first contact surface is elevated relative to the second contact surface.

Due to the asymmetric nature of the stapler head structure, structural means may be provided in order to improve firmness and/or structural stability (e.g., stability to torsion, bending and/or shearing forces). In designing such structural means, thought should also be made to the different compression pressures generated between first stapling jaw portion 130 and first contact surface 136 and between second stapling jaw portion 131 and second contact surface 137 during compression and stapling. As shown, blade 107 adds firmness to the stapling head, especially as it further advances distally, due to its two points connection to drive member 106 and anvil jaw 103 at driver-blade joint 110 and blade-anvil junction 111, respectively, with stapling jaw 104 arrested therebetween. Nevertheless, blade 107 is off-centered and closer to first stapling jaw portion 130, therefore a support 139 is also provided between the jaws, off-centered towards second stapling jaw portion 131 in a prescribed proportion to diminish torsions upon tissue compression and stapling.

A further aspect of the present invention provides a method of surgically fastening or/and cutting a bodily tissue or organ inside a body of a subject. The method may include at least one of the following steps (not necessarily in same order):
providing a linear tissue stapler head inside the body of the subject, the linear tissue stapler head comprises a stapling jaw configured to include a first stapling jaw portion and a second stapling jaw portion being relatively positioned so as to be oppositely separated from each other by a longitudinal channel extending therebetween;
clasping the bodily tissue or organ between the stapling jaw and an anvil jaw;
passing a blade having a sharp edge along the longitudinal channel to form a cutting line across the bodily tissue or organ; and
actuating the stapler head such that the first stapling jaw portion releases a first row of tissue fasteners of a first staple size or/and type along and adjacent a first side of the cutting line, and the second stapling jaw portion releases a second row of tissue fasteners of a second staple size or/and type along and adjacent a second side of the cutting line.

According to yet a further aspect of the present invention there is provided a method of surgically fastening and cutting a bodily tissue or organ inside a body. The method may include at least one of the following steps (not necessarily in same order):
providing a linear tissue stapler head inside the body of the subject, the linear tissue stapler head comprises a stapling jaw configured to include a first stapling jaw portion and a second stapling jaw portion being relatively positioned so as to be oppositely separated from each other by a longitudinal channel extending therebetween;
clasping the bodily tissue or organ between the stapling jaw and an anvil jaw; and
actuating the stapler head thereby dividing the bodily tissue or organ into a first body part having a first cut and sealed end and a second body part having a second cut and sealed end, the first cut and sealed end is held by tissue fasteners of a first size or/and type and the second cut and sealed end is held by tissue fasteners of a second size or/and type.

According to yet another aspect, the present invention provides a method of surgically fastening and cutting a bodily tissue or organ inside a body. The method may include at least one of the following steps (not necessarily in same order):
providing a passage connecting between an ex-vivo environment and an in-vivo location in vicinity of the bodily tissue or organ;
passing a linear tissue stapler through the passage towards the in-vivo location, the linear tissue stapler comprises an elongated body having a longitudinal axis and a stapler head, wherein the stapler head comprises a first member configured for including a plurality of parallel rows holding tissue fasteners, a second member, and a drive member including a blade having a sharp edge extending between the first and second members;
effecting emergence, via the passage, of the stapler head out of the body; and
operating the linear tissue stapler so as to effect shifting of the blade from a horizontal position to a vertical position, relative to the longitudinal axis, and to effect releasing of the tissue fasteners from the first member, thereby fastening and cutting the bodily organ or tissue.

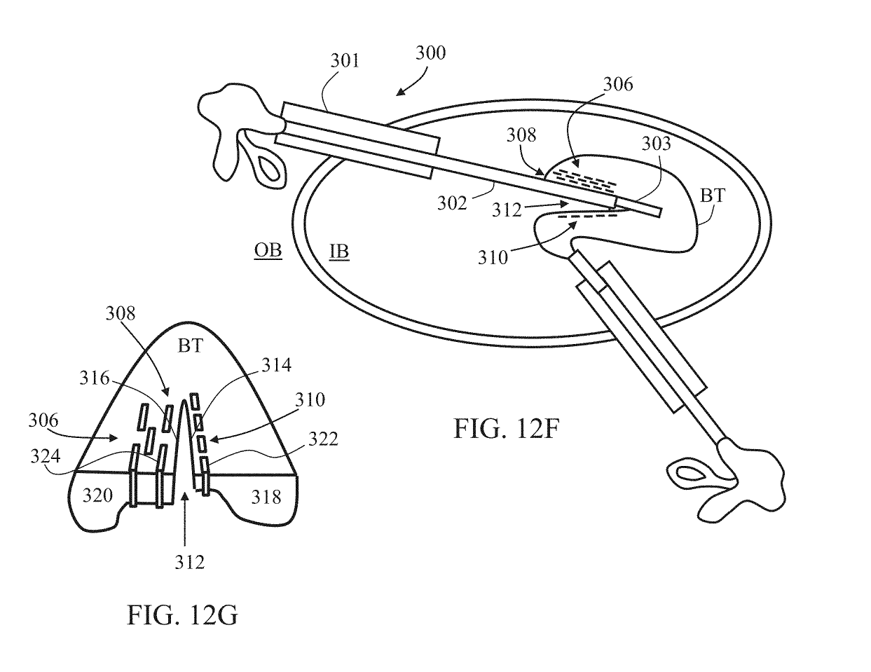

Reference is now made to FIGS. 12A-12G which schematically illustrate different scenarios representing possible exemplary steps in a method of deploying a linear tissue stapler 300 and surgically affecting a bodily tissue or/and organ, such as tissue BT, in a body, in accordance with some embodiments of the present invention. Linear tissue stapler 300 may be similar or identical to any of previously described staplers 1, 30, 40, 50, 60, 70, 100, 200 and 220. As shown in FIG. 12A, passage 301 (optionally enclosed with a laparoscopic port) is provided for connecting between an outer body environment OB and an in-body location IB in vicinity of tissue BT. As shown in FIG. 12B, linear tissue stapler 300 is passed through passage 301 towards in-vivo (in-body) location IB. Linear tissue stapler 300 comprises an elongated body 302 and is arranged in a delivery configuration in which a stapling jaw 303 is positioned distally to elongated body 302 and anvil jaw 304 is consecutively arranged lengthwise behind stapling jaw 303 relative to elongated body 302.

Stapling jaw 303 and anvil jaw 304 can then be emerged out of passage 301 within the body, allowing for linear tissue stapler 300 to be converted (inside the body) from the delivery configuration to a deployment configuration (as shown in FIGS. 12C-12D), whereby anvil jaw 304 repositions to oppose stapling jaw 303 so as to effect stapling jaw 303 and anvil jaw 304 to be juxtapositionally arranged, one beneath the other, and interlocked with each other to rigidly form stapling jaw 305. The linear tissue stapler can be operated following converting the linear tissue stapler from the delivery configuration to the deployment configuration.

FIGS. 12E and 12F show tissue BT being clasped between stapling jaw 303 and anvil jaw 304, and stapler 300 is employed such that stapling jaw 303 releases a plurality of tissue fasteners (e.g., staples) through tissue BT towards anvil jaw 304. A blade (e.g., similar to blade 107 in FIG. 11A) having a sharp edge may then be passed along a longitudinal channel, provided in the stapler head of stapler 300, to form a cutting line 312 across tissue BT. Stapler head of stapler 300 can be actuated (in a way that follows or includes blade passing/travel) such that a first stapling jaw portion thereof releases a first row 310 of tissue fasteners of a first staple size or/and type along and adjacent a first side 314 of cutting line 312, and a second stapling jaw portion thereof releases a second row 308 of tissue fasteners of a second staple size or/and type along and adjacent a second side 316 of cutting line 312. In some embodiments and as shown, second row 308 may be part of a plurality of rows 306 of tissue staples, not necessarily of second or first staple size or/and type. Optionally, tissue fasteners of first size or/and type (provided in and along first row 310 of tissue fasteners) or/and of second size or/and type (provided in and along second row 308 or/and remaining rows 306 of tissue fasteners) have staple legs that are plastically deformable to an inwardly bent form.

According to some embodiments, passing the blade and releasing the first row 310 and second row 308 of tissue fasteners effects dissecting of the tissue BT along cutting line 312 into a removable body part 318 sealed with first row 310 of tissue fasteners, and a remaining body part 320 sealed with second row 308 or/and remaining rows 306 of tissue fasteners. FIG. 12G illustrates a transverse cut of tissue BT, partially cut and sealed/stapled (stapler 300 and parts thereof are absent), from a different view point suggesting a possible exemplary effect of the methods of invention. As shown, removable body part 318 is compressed via first row 310 of tissue fasteners, and remaining body part 320 is compressed (distinctly and independently to removable body part 318 compressing) via second row 308 (or/and via remaining rows 306) of tissue fasteners, such that, adjacent the cutting line 312, removable body part 318 has a smaller thickness than the remaining body part 320. Different thicknesses result from different sizes (or/and types) of tissue fasteners between first 310 and second 308 rows, optionally and particularly by staple leg-size differentiations. Optionally, tissue fasteners of second size or/and type (provided in and along second row 308 or/and remaining rows 306 of tissue fasteners) have a minimal leg length being greater than maximal leg length of the tissue fasteners of the first size or/and type. An optional minimal leg size of the second tissue fastener may be, for example, greater than about 3 mm in length, optionally about 3.5 mm, or optionally about 3.8 mm, or optionally about 4.1 mm, in length. In contrast, a leg size of the first tissue fastener may be smaller than about 3 mm in length, optionally about 2.5 mm or optionally about 2 mm. Optionally, additionally or alternatively, a minimal leg size of second tissue fasteners legs is optionally equal to or greater than about 1.5 mm, optionally equal to or greater than about 2 mm, in length, whereas a maximal leg size of first tissue fasteners legs is optionally smaller by at least 0.2 mm, optionally by at least 0.5 mm, optionally by at least 1 mm, optionally by at least 1.5 mm, than the minimal leg size of second tissue fasteners.

Optionally, alternatively or additionally, length of staples bridge 322 of the first size or/and type is smaller than length of staples bridge 324 of the second size or/and type, or/and optionally distance between tissue fasteners along first row 310 is smaller than distance between tissue fasteners along second row 308, optionally resulting in stacking of a greater number of tissue fasteners in first row 310 than in second row 308 of tissue fasteners.

Each of the following terms written in singular grammatical form: ‘a’, ‘an’, and ‘the’, as used herein, means ‘at least one’, or ‘one or more’. Use of the phrase ‘one or more’ herein does not alter this intended meaning of ‘a’, ‘an’, or ‘the’. Accordingly, the terms ‘a’, ‘an’, and ‘the’, as used herein, may also refer to, and encompass, a plurality of the stated entity or object, unless otherwise specifically defined or stated herein, or, unless the context clearly dictates otherwise. For example, the phrases: ‘a unit’, ‘a device’, ‘an assembly’, ‘a mechanism’, ‘a component’, ‘an element’, and ‘a step or procedure’, as used herein, may also refer to, and encompass, a plurality of units, a plurality of devices, a plurality of assemblies, a plurality of mechanisms, a plurality of components, a plurality of elements, and, a plurality of steps or procedures, respectively.

Each of the following terms: ‘includes’, ‘including’, ‘has’, ‘having’, ‘comprises’, and ‘comprising’, and, their linguistic/grammatical variants, derivatives, or/and conjugates, as used herein, means ‘including, but not limited to’, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof. Each of these terms is considered equivalent in meaning to the phrase ‘consisting essentially of.’ Each of the phrases ‘consisting of’ and ‘consists of’, as used herein, means ‘including and limited to.’ The phrase ‘consisting essentially of’, as used herein, means that the stated entity or item (system, system unit, system sub-unit, device, assembly, sub-assembly, mechanism, structure, component, element, or, peripheral equipment, utility, accessory, or material, method or process, step or procedure, sub-step or sub-procedure), which is an entirety or part of an exemplary embodiment of the disclosed invention, or/and which is used for implementing an exemplary embodiment of the disclosed invention, may include at least one additional ‘feature or characteristic’ being a system unit, system sub-unit, device, assembly, sub-assembly, mechanism, structure, component, or element, or, peripheral equipment, utility, accessory, or material, step or procedure, sub-step or sub-procedure), but only if each such additional ‘feature or characteristic’ does not materially alter the basic novel and inventive characteristics or special technical features, of the claimed entity or item.

The term ‘method’, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed invention.

Throughout this disclosure, a numerical value of a parameter, feature, characteristic, object, or dimension, may be stated or described in terms of a numerical range format. Such a numerical range format, as used herein, illustrates implementation of some exemplary embodiments of the invention, and does not inflexibly limit the scope of the exemplary embodiments of the invention. Accordingly, a stated or described numerical range also refers to, and encompasses, all possible sub-ranges and individual numerical values (where a numerical value may be expressed as a whole, integral, or fractional number) within that stated or described numerical range. For example, a stated or described numerical range ‘from 1 to 6’ also refers to, and encompasses, all possible sub-ranges, such as ‘from 1 to 3’, ‘from 1 to 4’, ‘from 1 to 5’, ‘from 2 to 4’, ‘from 2 to 6’, ‘from 3 to 6’, etc., and individual numerical values, such as ‘1’, ‘1.3’, ‘2’, ‘2.8’, ‘3’, ‘3.5’, ‘4’, ‘4.6’, ‘5’, ‘5.2’, and ‘6’, within the stated or described numerical range of ‘from 1 to 6’. This applies regardless of the numerical breadth, extent, or size, of the stated or described numerical range.

Moreover, for stating or describing a numerical range, the phrase ‘in a range of between about a first numerical value and about a second numerical value’, is considered equivalent to, and meaning the same as, the phrase ‘in a range of from about a first numerical value to about a second numerical value’, and, thus, the two equivalently meaning phrases may be used interchangeably. For example, for stating or describing the numerical range of room temperature, the phrase ‘room temperature refers to a temperature in a range of between about 20° C. and about 25° C.’, and is considered equivalent to, and meaning the same as, the phrase ‘room temperature refers to a temperature in a range of from about 20° C. to about 25° C.’

The term ‘about’, as used herein, refers to ±10% of the stated numerical value.

It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub combination in the context or format of a single embodiment, may also be illustratively described and presented in the context or format of a plurality of separate embodiments.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A tissue stapler head, comprising:
a stapling jaw configured to include a first stapling jaw portion and a second stapling jaw portion being relatively positioned so as to be oppositely separated from each other by a longitudinal channel extending therebetween, said longitudinal channel is sized and configured for allowing a blade having a sharp edge to travel therealong; and
an anvil jaw positionable to oppose said stapling jaw;
wherein said first stapling jaw portion includes a first row of tissue fasteners of a first staple size or/and type adjacent to a first side of said longitudinal channel, and said second stapling jaw portion includes a second row of tissue fasteners of a second staple size or/and type adjacent to a second side of said longitudinal channel.

2. The tissue stapler head according to claim 1, wherein said tissue fasteners of said second size or/and type have staple legs that are plastically deformable to an inwardly bent form.

3. The tissue stapler head according to claim 1, wherein said tissue fasteners of said second size or/and type have a minimal leg length being greater than about 3 mm.

4. The tissue stapler head according to claim 1, wherein said tissue fasteners of said first size or/and type have a maximal leg length being less than about 3 mm.

5. The tissue stapler head according to claim 1, wherein at least one of said tissue fasteners of said first and second size or/and type includes at least one rigid or elastic prong.

6. The tissue stapler head according to claim 1, wherein said second stapling jaw portion has a plurality of rows of tissue fasteners including said second row of tissue fasteners of said second staple size or/and type.

7. The tissue stapler head according to claim 1, wherein said first stapling jaw portion includes suturing means.

8. The tissue stapler head according to claim 1, wherein said first stapling jaw portion includes tissue bonding means, tissue coagulating means, or tissue adhering means.

9. The tissue stapler head according to claim 1, wherein, in close proximity between said stapling jaw and said anvil jaw, said first stapling jaw portion faces a first contact surface of said anvil jaw and said second stapling jaw portion faces a second contact surface of said anvil jaw, wherein a first distance extending between said first stapling jaw portion and said first contact surface is less than a second distance extending between said second stapling jaw portion and said second contact surface.

10. The tissue stapler head according to claim 9, wherein said first contact surface and said second contact surface are distanced apart from each other by a contact surface transition portion located therebetween, said contact surface transition portion includes a rise such that said first contact surface is elevated relative to said second contact surface.

11. The tissue stapler head according to claim 1, configured and operative so as to be convertible from a delivery configuration including said stapler head in a non-operational mode and in which said stapler head is passable through a passage enclosing a minimal inner diameter, to a deployment configuration including said stapler head being in an operational mode.

12. The tissue stapler head according to claim 11, wherein, in said delivery configuration, said stapling jaw and said anvil jaw are consecutively arranged lengthwise relative to each other.

13. The tissue stapler head according to claim 11, wherein, in said delivery configuration, said stapler head has maximal cross-sectional dimensions including a maximal height, a maximal width, or/and a maximal diameter, each of said dimensions being smaller than said minimal inner diameter of said passage.

14. The tissue stapler head according to claim 13, wherein, in said delivery configuration, each of said maximal stapler head cross-sectional dimensions is equal to or less than about 8 mm.

15.-41. (canceled)
